(19) United States
(12) Patent Application Publication
(10) Pub. No.: US 2023/0121184 A1
(43) Pub. Date: Apr. 20, 2023

(54) ORGANIC SEMICONDUCTING COMPOUND AND THE ORGANIC PHOTOELECTRIC COMPONENTS USING THE SAME

(71) Applicant: RAYNERGY TEK INCORPORATION, HSINCHU (TW)

(72) Inventors: YU-TANG HSIAO, HSINCHU (TW); WEI-LONG LI, HSINCHU (TW); CHIA-HUA TSAI, HSINCHU (TW); CHUANG-YI LIAO, HSINCHU (TW)

(21) Appl. No.: 17/895,722

(22) Filed: Aug. 25, 2022

Related U.S. Application Data
(60) Provisional application No. 63/237,722, filed on Aug. 27, 2021.

(51) Int. Cl. H01L 51/00; C07D 519/00

(52) U.S. Cl. CPC H01L 51/0068; C07D 519/00; H01L 51/0071; H01L 51/0074; H01L 51/0069; H01L 51/424

(57) ABSTRACT

The present invention relates to an Organic Semiconducting Compound and organic photoelectric components using the same. The innovative chemical structure of the Organic Semiconducting Compound allows improved infrared light range response values and renders it suitable for uses in the organic photoelectric components, such as OPD, OFET, or OPV due to its broadened absorbance wavelength range and improved external quantum efficiency.

### FIELD OF THE INVENTION

The subject invention relates to a compound and photoelectronic components containing the subject compound. This compound comes with especially good physical and chemical properties, which can also be processed with an environment-friendly organic solvent to enhance the convenience for the production of the organic semiconducting compound that makes a lower impact on the environment, as well as organic photoelectronic components with excellent response values in the infrared range.

### BACKGROUND OF THE INVENTION

In recent years, them has been an increasing demand for manufacturing more versatile and lower-cost electronic components; hence, the increasing demand for organic semiconducting compounds (OSC). The reason behind this phenomenon is mainly due to the wider light absorption range, higher light absorption coefficient, and adjustable structure of the organic semiconducting compound, in comparison with the traditional semiconductor materials. In terms of the light absorption range, energy level, and solubility, they can all be adjusted according to the target requirements. In addition, in the aspect of manufacturing the organic components, organic materials have the advantages of low cost, flexibility, low toxicity, and suitability for mass production, making organic photoelectronic materials high competitiveness in various fields. Such compounds can be applied in a wide range, including organic field-effect transistors (OFETs), organic light-emitting diodes (OLEDs), organic photodetectors (OPDs), organic photovoltaics (OPVs), sensors, storage components, and various components or assemblies of logic circuits. Among them, organic semiconductor materials usually exist in the form of thin layers in the above-mentioned applications of components or assemblies, with a thickness of about 50 nm to 1 μm.

Organic photodetector (OPD) makes an emerging field of organic photoelectronics in recent years. Such devices can detect various light sources in the environment and are used in application fields such as medical care, health management, intelligent driving, unmanned aerial vehicle, digital home, etc. Therefore, there are different material requirements for different application fields. Moreover, the use of organic materials enables the device with good flexibility. Benefit from the development of material science, OPD can not only be made into a thin layer but also can absorb light in specific wavelength bands. According to the different light sources, products on the market need to absorb light in different bands. Therefore, the use of organic materials can adjust the absorption range, which can effectively absorb the required wavelength bands to reduce interference, and the high extinction coefficient of organic materials can also effectively improve the detection efficiency. In recent years, the development of OPD has covered from ultraviolet and visible light to near-infrared (NIR).

In terms of performance, the material of the active layer in the organic photodetector directly contributes to the performance of the device, therefore the active layer plays an important role. The materials of the active layer consist of the donor and the acceptor. On the one hand, common donor materials include organic polymers, oligomers, or limited molecular units. Nowadays D-A type conjugate polymers make the mainstream among donors. Through the interaction between the electron-rich unit and the electron-deficient unit in the polymer, the push-pull electron effect formed can be used to control the energy level and energy gap of the polymer. On the other hand, the matching acceptor materials are usually fullerene derivatives with high conductivity, with a light absorption range of about 400˜600 nm. Additionally, graphene, metal oxides, or quantum dots (QDs) are also included. However, the structure of fullerene derivatives is hard to adjust, and the range of absorption wavelength bands and energy levels are limited; thus, limiting the overall matching of donor and acceptor materials. With the development of the market, the demand for materials within the near-infrared range is gradually increasing. Even though the light absorption range of the conjugate polymer donor can be adjusted to the near-infrared range, it may not be well matched due to the limit of the fullerene acceptors. In the end, the development of non-fullerene acceptor compounds to replace the traditional fullerene acceptors becomes especially important in the breakthrough of active layer materials.

Nonetheless, the development of non-fullerene acceptor compounds was quite difficult in the early stages because it was not easy to control the morphology of the compound, hence resulting in low power conversion efficiency. However, since 2015, numerous studies on non-fullerene acceptors have significantly improved their electrical performance, thus making non-fullerene acceptors a competitive choice. This change is mainly attributable to the advancement of synthesis methods and the improvement of material design strategies, etc. The wide range of donor materials previously developed for matching the fullerene acceptors has also indirectly contributed to the development of non-fullerene acceptor compounds.

At present, the development of non-fullerene acceptor compound materials mainly lies in the molecular structure of A-D-A mode which consists of one electron-rich center unit with two electron-deficient units, where D is usually a molecule composed of benzene ring and thiophene, and A is usually an indanone-cyano (IC) derivatives. Another mode of molecular structure is the A′-D-A-D-A′ mode with the center of one electron-deficient unit where a molecule such as sulfur atom is often used therewith to enhance the performance.

In the field of intelligent driving and an unmanned aerial vehicle, to avoid visible light with a too strong signal, the development trend is to adopt the NIR as the absorption band. Moreover, to have the properties of better penetration and long-distance detection, the application wavelength needs to exceed 1,000 nm. Besides, in response to the requirements of environmental protection regulations and good processing operability in various countries, environmentally friendly solvents must be used as much as possible during the material process, to be conducive to wet process operations. Currently, the organic semiconducting materials with the relevant potentials are polymers either with a donor-acceptor or a small molecule structure and only perform well in the absorbance range <1000 nm, whereas the overall component performance of materials >1000 nm is poor; moreover, the solvents used in solution process are mainly organic solvents containing halogen and have a great impact on the environment. Therefore, the need is imminent to develop an organic semiconducting compound that has superior photo-response performance in the infrared range, and uses halogen-free organic solvents during the solution process.

### SUMMARY OF THE INVENTION

Given the problems about the deficiencies of current materials in the preceding paragraphs, the objective of the subject invention is to provide a new organic semiconducting compound, especially an n-type organic semiconducting compound, which can overcome the shortcomings of the organic semiconducting compounds based on the previous technologies, while providing one or more advantageous properties discussed above, including a photo-response greater than 1,000 nm and good device performance, as well as easy synthesis demonstrated during the production process, such as good processability and good solubility in environmentally-friendly solvents, thus facilitating the large-scale manufacturing, i.e. mass production, with the solution processing method.

Another object of the invention is to provide a new organic photoelectric component comprising the Organic Semiconducting Compound of the invention, its photo-responsivity is greater than 1000 nm and has excellent external quantum efficiency.

To achieve the purpose stated above, the invention provides an Organic Semiconducting Compound, which is represented by the following formula:

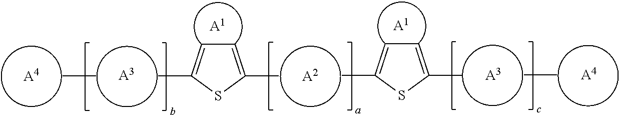

Wherein,
A¹ is selected from the group composed of the following groups:

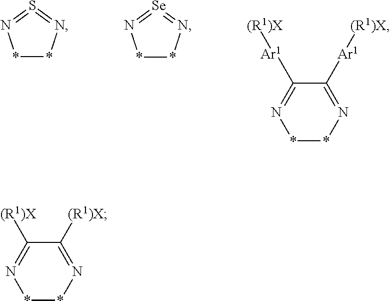

x is an integer between 0 and 5;
Ar¹ is aromatic ring or heteroaromiatic ring group, which is monocyclic or polycyclic, and is unsubstituted or substituted with halogen atoms;
R¹ is selected from the group consisting of the following groups: hydrogen atom, halogen, cyano group, C1˜C30 linear alkyl, C3˜C30 branched alkyl, C1˜C30 silyl group, C2˜C30 ester group, C1˜C30 alkoxy, C1˜C30 thioalkyl, C1˜C30 haloalkyl, C2˜C30 alkene, C2˜C30 alkyne, C2˜C30 cyano-substituent alkyl, C1˜C30 nitro-substituent alkyl, C1˜C30 hydroxy-substituent alkyl, C3˜C30 keto-substituent alkyl;
A²˜A⁴ are each aromatic ring or heteroaromatic ring group which is monocyclic or polycyclic; and
a, b, and c are an integer between 0 and 5.

To achieve the objective mentioned above, the subject invention further provides an organic photoelectronic component, which comprises: a substrate; an electrode module disposed on the substrate, which includes a first electrode and a second electrode; an active layer disposed between the first electrode and the second electrode, where the active layer includes at least one organic semiconducting compound as described in the present invention; where at least one of the first electrode and the second electrode is transparent or semi-transparent.

### BRIEF DESCRIPTION OF DRAWINGS

FIG. 1A-IF are schematic diagrams of the structure of the organic photoelectronic components of the subject invention;

FIG. 2A-2C are graphs of experimental results for the organic photoelectronic components of the subject invention;

FIG. 3A-3B are graphs of experimental results for the organic photoelectronic components of the subject invention;

FIG. 4A-4B are graphs of experimental results for the organic photoelectronic components of the subject invention;

FIG. 5A-5B are graphs of experimental results for the organic photoelectronic components of the subject invention;

FIG. 6A-6B are graphs of experimental results for the organic photoelectronic components of the subject invention; and

FIG. 7A-7B are graphs of experimental results for the organic photoelectronic components of the subject invention.

### DESCRIPTION OF EMBODIMENTS

The organic semiconducting compound under the subject invention not only possesses the characteristic of easy synthesis but also exhibits good processability and solubility in common solvents during the production process, thus facilitating the large-scale manufacturing during the solution process.

The preparation of the organic semiconducting compound under the subject invention can be achieved based on the methods known to those with ordinary knowledge in the technical field covering the subject invention and described in the literature, which will be further illustrated in the experimental examples.

The Organic Semiconducting Compound provided by the invention is represented by the following formula:

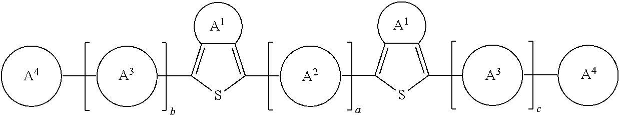

Wherein,
A¹ is a group selected from the following groups.

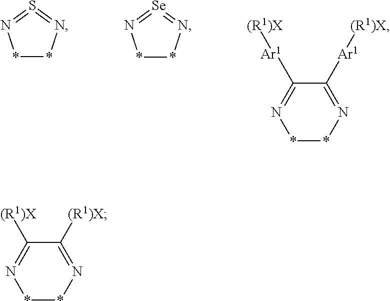

x is an integer between 0 and 5;
Ar¹ is aromatic ring or heteroaromatic ring group, which is monocyclic or polycyclic, and is unsubstituted or substituted with halogen atoms;
R¹ is selected from the group consisting of the following groups: hydrogen atom, halogen, cyano group, C1˜C30 linear alkyl, C3˜C30 branched alkyl, C1˜C30 silyl group, C2˜C30 ester group, C1˜C30 alkoxy, C1˜C30 thioalkyl, C1˜C30 haloalkyl, C2˜C30 alkene, C2˜C30 alkyne, C2˜C30 cyano-substituent alkyl, C1˜C30 nitro-substituent alkyl, C1˜C30 hydroxy-substituent alkyl, C3˜C30 keto-substituent alkyl;
A²˜A⁴ are each aromatic ring or heteroaromatic ring group which is monocyclic or polycyclic; and
a, b, and c are an integer between 0 and 5,

In the formula for the organic semiconducting compound under the subject invention, the aromatic ring of Ar¹ preferably has 4 to 30 ring C atoms, which is monocyclic or polycyclic, and may also contain fused rings, preferably with 1, 2, 3, 4 or 5 fused or unfused rings, and optionally with one or more halogen-substituents.

In the formula for the organic semiconducting compound under the subject invention, the heteroaromatic ring of Ar¹ preferably has 4 to 30 ring C atoms, where one or more ring C atoms are heteroatoms preferably selected from N, O, S, Si, and Se substituent, which is monocyclic or polycyclic, and may also contain fused rings, preferably 1, 2, 3, 4 or 5 fused or unfused rings, and optionally with one or more halogen-substituents.

In the formula for the organic semiconducting compound under the subject invention, R¹ can be alkyl or alkoxy (i.e. one of the CH₂ groups is substituted with —O—), linear chain or branched chain. Particularly preferred linear chains come with 2, 3, 4, 5, 6, 7, 8, 12, or 16 carbon atoms, therefore are the preferred ethyl, propyl, butyl, pentyl, hexyl, heptyl, octyl, dodecyl, or hexadecyl; or ethoxy, propoxy, butoxy, pentyloxy, hexyloxy, heptyloxy, octyloxy, dodecyloxy or hexadecyloxy; or methyl, nonyl, decyl, undecyl, tridecyl, tetradecyl, or pentadecyl; or nonyloxy, decyloxy, undecyloxy, tridecyloxy or tetradecyloxy.

In the formula for the organic semiconducting compound under the subject invention, R¹ can be alkenyl (i.e. one or more CH₂ groups in the alkyl group are substituted with —CH═CH—), linear chain or branched chain. Particularly preferred linear chain come with 2 to 10 C atoms, therefore are the preferred vinyl; propen-1-, 2-yl; buten-1-, 2- or 3-yl; penten-1-, 2-, 3- or 4-yl; hexen-1-, 2-, 3-, 4- or 5-yl; heptene-1-, 2-, 3-, 4-, 5- or 6-yl; octen-1-, 2-, 3-, 4-, 5-, 6- or 7-yl; nonen-1-, 2-, 3-, 4-, 5-, 6-, 7- or 8-yl; or decene-1-, 2-, 3-, 4-, 5-, 6-, 7-, 8- or 9-yl.

In the formula for the organic semiconducting compound under the subject invention, R can preferably be thioalkyl (i.e. one of the CH₂ groups is substituted with —S—). Particularly preferred linear chain include thiomethyl (—SCH₃), 1-thioethyl (—SCH₂CH₃), 1-thiopropyl (—SCH₂CH₂CH₃), 1-(thiobutyl), 1-(thiopentyl), 1-(thiohexyl), 1-(thioheptyl), 1-(thiooctyl), 1-(thiononyl), 1-(thiodecyl), 1-(thioundecyl) or 1-(thiododecyl), wherein preferably the CH₂ group adjacent to the sp² hybridized vinyl carbon atom is replaced.

In the formula for the organic semiconducting compound under the subject invention, the halogen of in R¹ can include F, Cl, Br or I.

In a preferred example of the invention, A² of the Organic Semiconducting Compound is selected from the group composed of the following groups:

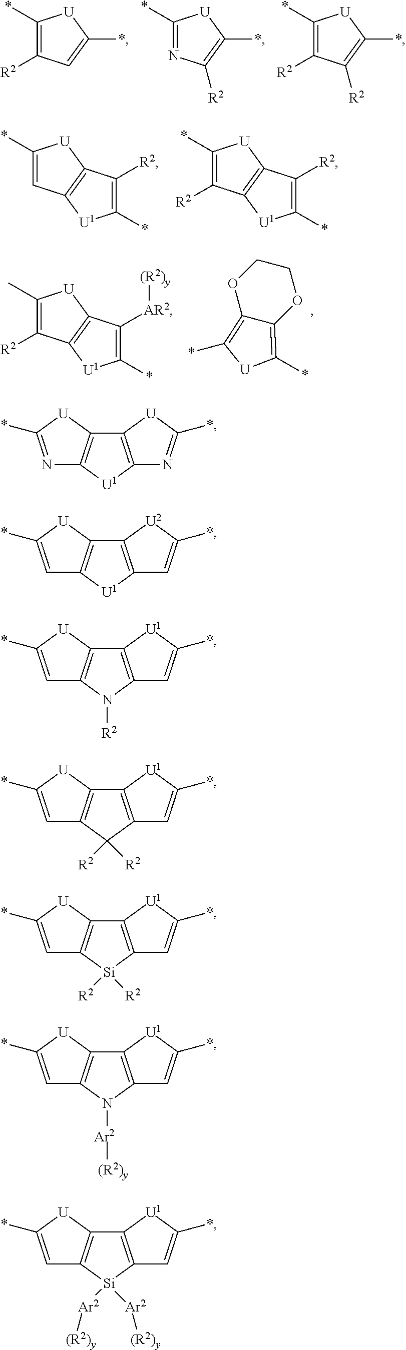

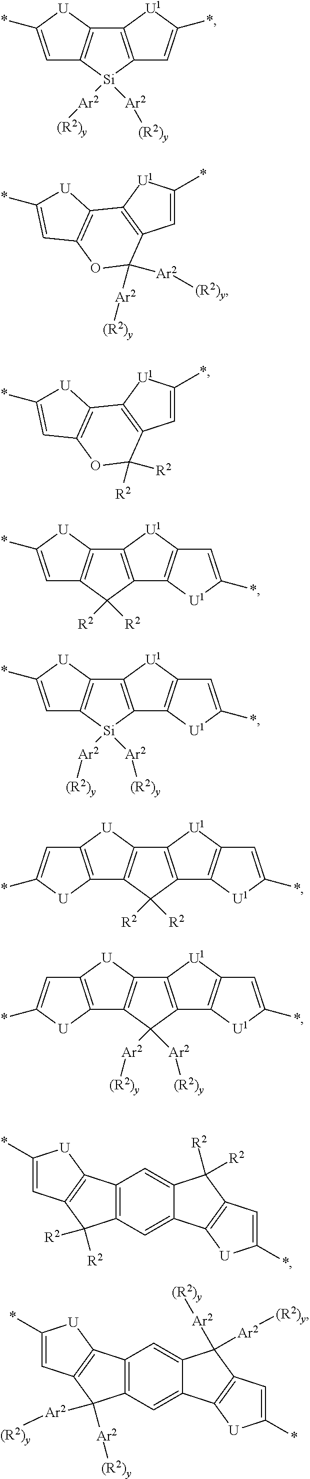

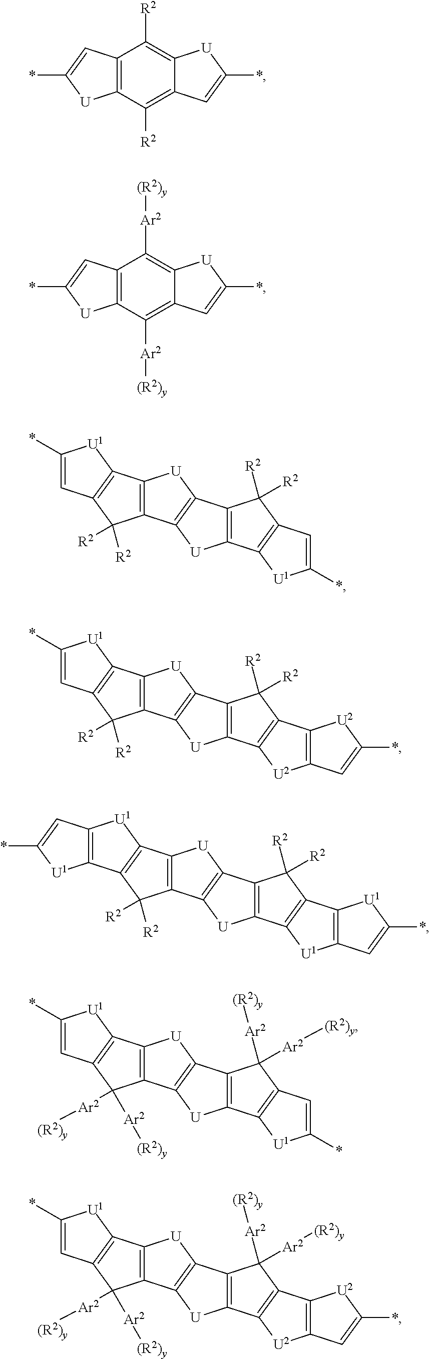

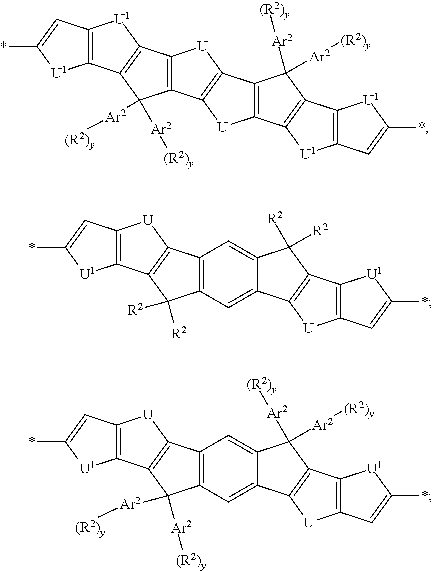

Wherein,

U, U¹, and U² are selected among O, S, and Se,

y is an integer between 0 and 5;

Ar² is aromatic ring or heteroaromatic ring group, which is monocyclic or polycyclic, and is unsubstituted or substituted with halogen atoms; and

R² is selected from the group consisting of the following groups: hydrogen atom, halogen, cyano group, C1˜C30 linear alkyl, C3˜C30 branched alkyl, C1˜C30 silyl group, C2˜C30 ester group, C1˜C30 alkoxy, C1˜C30 thioalkyl, C1˜C30 haloalkyl, C2˜C30 alkene, C2˜C30 alkyne, C2˜C30 cyano-substituent alkyl, C1˜C30 nitro-substituent alkyl, C1˜C30 hydroxy-substituent alkyl, C3˜C30 keto-substituent alkyl;

In the formula for the organic semiconducting compound under the subject invention, the aromatic ring of Ar² preferably has 4 to 30 ring C atoms, which are monocyclic or polycyclic, and may also contain fused rings, preferably with 1, 2, 3, 4 or 5 fused or unfused rings, and optionally with one or more halogen-substituents.

In the formula for the organic semiconducting compound under the subject invention, the heteroaromatic ring of Ar² preferably has 4 to 30 ring C atoms, where one or more ring C atoms are heteroatoms preferably selected from N, O, S, Si and Substituent, which is monocyclic or polycyclic, and may also contain fused rings, preferably 1, 2, 3, 4 or 5 fused or unfused rings, and optionally with one or more halogen-substituents.

In the formula for the organic semiconducting compound under the subject invention, R² can be alkyl or alkoxy (i.e. one of the CH₂ groups is substituted with —O—), linear chain or branched chain. Particularly preferred linear chain come with 2, 3, 4, 5, 6, 7, 8, 12 or 16 carbon atoms, therefore are the preferred ethyl, propyl, butyl, pentyl, hexyl, heptyl, octyl, dodecyl, or hexadecyl; or ethoxy, propoxy, butoxy, pentyloxy, hexyloxy, heptyloxy, octyloxy, dodecyloxy or hexadecyloxy; or methyl, nonyl, decyl, undecyl, tridecyl, tetradecyl, or pentadecyl; or nonyloxy, decyloxy, undecyloxy, tridecyloxy or tetradecyloxy.

In the formula for the organic semiconducting compound under the subject invention, R² can be alkenyl (i.e. one or more CH₂ groups in the alkyl group are substituted with —CH═CH—), linear chain or branched-chain. Particularly preferred linear chain come with 2 to 10 C atoms, therefore are the preferred vinyl; propen-1-, 2-yl; buten-1-, 2- or 3-yl; Penten-1-, 2-, 3- or 4-yl; hexen-1-, 2-, 3-, 4- or 5-yl; heptene-1-, 2-, 3-, 4-, 5- or 6-yl; octen-1-, 2-, 3-, 4-, 5-, 6- or 7-yl; nonen-1-, 2-, 3-, 4-, 5-, 6-, 7- or 8-yl; or decene-1-, 2-, 3-, 4-, 5-, 6-, 7-, 8- or 9-yl.

In the formula for the organic semiconducting compound under the subject invention, R² can preferably be thioalkyl (i.e. one of the CH₂ groups is substituted with —S—). Particularly preferred linear chain include thiomethyl (—SCH₃), 1-thioethyl (—SCH₂CH₃), 1-thiopropyl (═—SCH₂CH₂CH₃), 1-(thiobutyl), 1-(thiopentyl), 1-(thiohexyl), 1-(thioheptyl), 1-(thiooctyl), 1-(thiononyl), 1-(thiodecyl), 1-(thioundecyl) or 1-(thiododecyl), wherein preferably the CH₂ group adjacent to the sp² hybridized vinyl carbon atom is replaced.

In the formula for the organic semiconducting compound under the subject invention, the halogen of in R² can include F, Cl, Br or I.

More preferably, A² is selected from the group composed of the following groups:

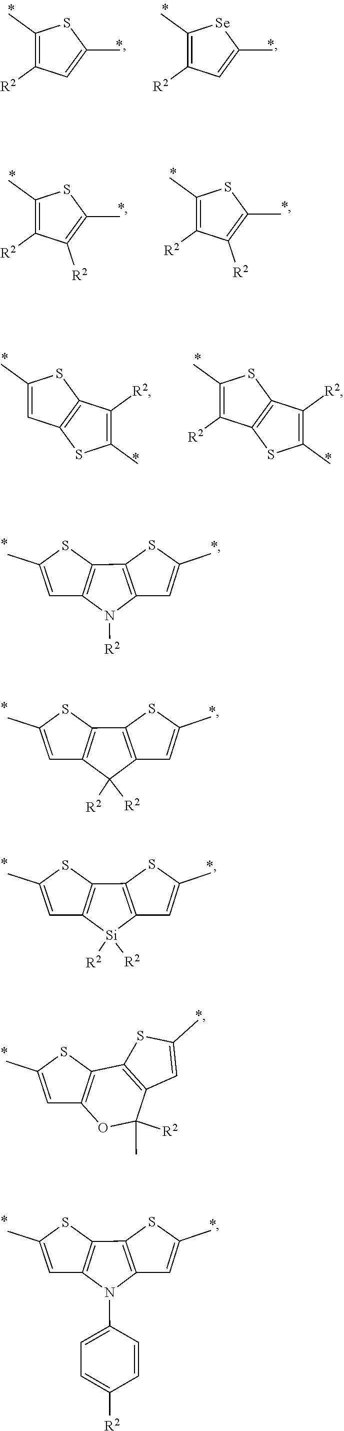

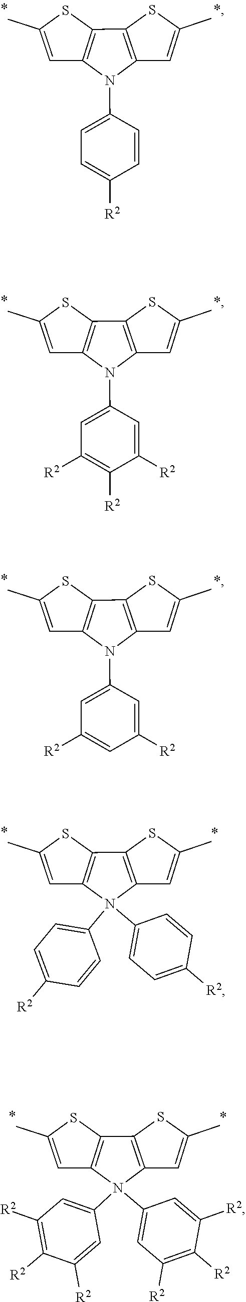

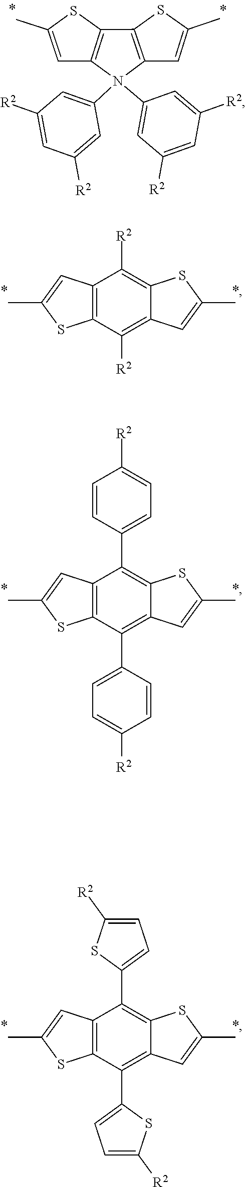

In a preferred example of the invention, A³ of the Organic Semiconducting Compound is selected frim the group composed of the following group:

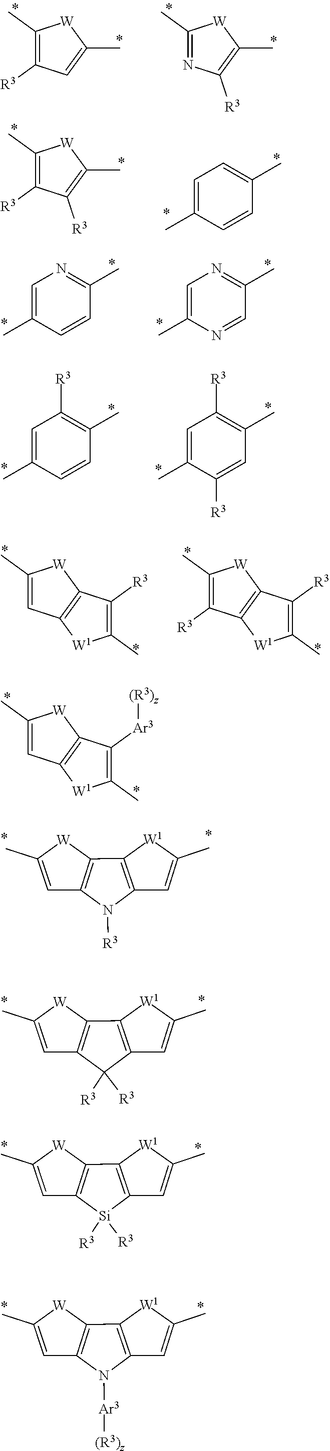

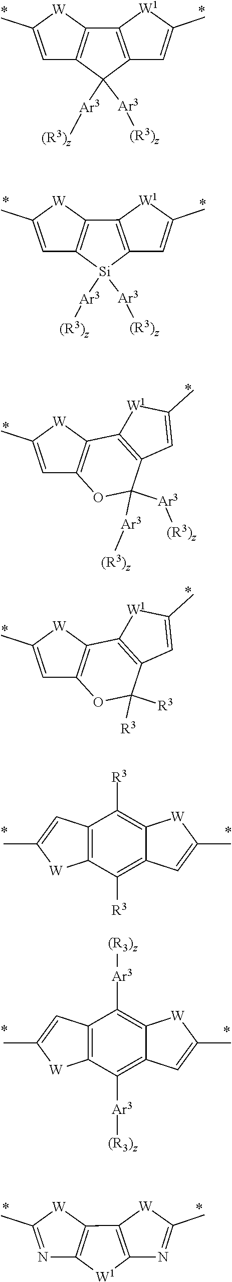

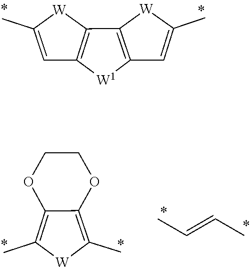

where W and W¹ are selected among O, S, and Se;
z is an integer between 0 and 5;
Ar³ is aromatic ring or heteroaromatic ring group, which is monocyclic or polycyclic, and is unsubstituted or substituted with halogen atoms; and
R³ is selected from the group consisting of the following groups: hydrogen atom, halogen, cyano group, C1˜C30 linear alkyl, C3˜C30 branched alkyl, C1˜C30 silyl group, C2˜C30 ester group, C1˜C30 alkoxy, C1˜C30 thioalkyl, C1˜C30 haloalkyl, C2˜C30 alkene, C2˜C30 alkyne, C2˜C30 cyano-substituent alkyl, C1˜C30 nitro-substituent alkyl, C1˜C30 hydroxy-substituent alkyl, C3˜C30 keto-substituent alkyl.

In the formula for the organic semiconducting compound under the subject invention, the aromatic ring of Ar³ preferably has 4 to 30 ring C atoms, which are monocyclic or polycyclic, and may also contain fused rings, preferably with 1, 2, 3, 4 or 5 fused or unfused rings, and optionally with one or more halogen-substituents.

In the formula for the organic semiconducting compound under the subject invention, the heteroaromatic ring of Ar³ preferably has 4 to 30 ring C atoms, where one or more ring C atoms are heteroatoms preferably selected from N, O, S, Si and Se substituent, which is monocyclic or polycyclic, and may also contain fused rings, preferably 1, 2, 3, 4 or 5 fused or unfused rings, and optionally with one or more halogen-substituents.

In the formula for the organic semiconducting compound under the subject invention, R³ can be alkyl or alkoxy (i.e. one of the CH₂ groups is substituted with —O—), linear chain or branched chain. Particularly preferred linear chain come with 2, 3, 4, 5, 6, 7, 8, 12 or 16 carbon atoms, therefore are the preferred ethyl, propyl, butyl, pentyl, hexyl, heptyl, octyl, dodecyl, or hexadecyl; or ethoxy, propoxy, butoxy, pentyloxy, hexyloxy, heptyloxy, octyloxy, dodecyloxy or hexadecyloxy; or methyl, nonyl, decyl, undecyl, tridecyl, tetradecyl, or pentadecyl; or nonyloxy, decyloxy, undecyloxy, tridecyloxy or tetradecyloxy.

In the formula for the organic semiconducting compound under the subject invention, R³ can be alkenyl (i.e. one or more CH₂ groups in the alkyl group are substituted with —CH═CH—), linear chain or branched-chain. Particularly preferred linear chain come with 2 to 10 C atoms, therefore are the preferred vinyl; propen-1-, 2-yl; buten-1-, 2- or 3-yl; Penten-1-, 2-, 3- or 4-yl; hexen-1-, 2-, 3-, 4- or 5-yl; heptene-1-, 2-, 3-, 4-, 5- or 6-yl; octen-1-, 2-, 3-, 4-, 5-, 6- or 7-yl; nonen-1-, 2-, 3-, 4-, 5-, 6-, 7- or 8-yl; or decene-1-, 2-, 3-, 4-, 5-, 6-, 7-, 8- or 9-yl.

In the formula for the organic semiconducting compound under the subject invention, R³ can preferably be thioalkyl (i.e. one of the CH₂ groups is substituted with —S—). Particularly preferred linear chain include thiomethyl (—SCH₃), 1-thioethyl (—SCH₂CH₃), 1-thiopropyl (═—SCH₂CH₂CH₃), 1-(thiobutyl), 1-(thiopentyl), 1-(thiohexyl), 1-(thioheptyl), 1-(thiooctyl), 1-(thiononyl), 1-(thiodecyl), 1-(thioundecyl) or 1-(thiododecyl), wherein preferably the CH2 group adjacent to the sp2 hybridized vinyl carbon atom is replaced.

In the formula for the organic semiconducting compound under the subject invention, the halogen of R³ of the invention comprises F, Cl, Br, or I.

More preferably, A³ is selected from the group composed of the following groups:

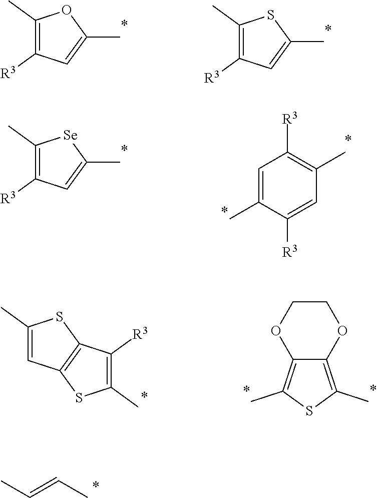

In a preferred example of the invention, A⁴ of the Organic Semiconducting Compound is selected from the group composed of the following groups:

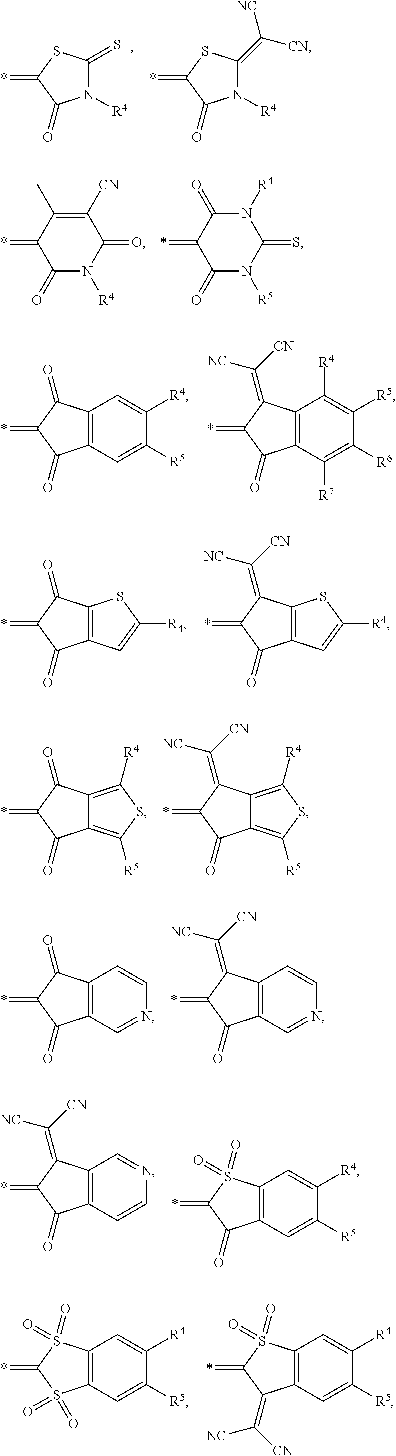

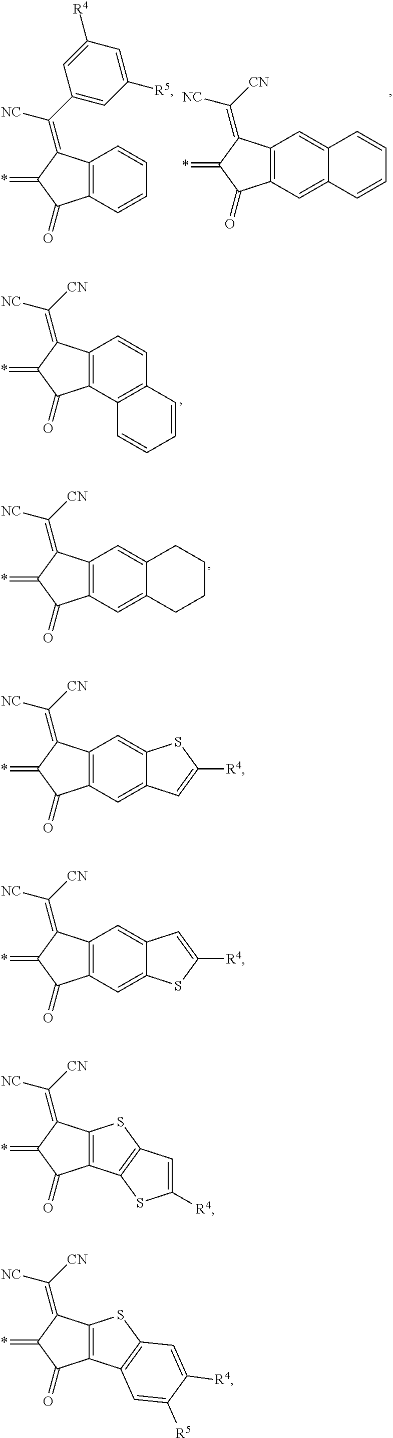

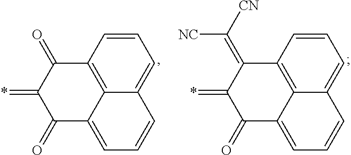 [SMILES: *=c1c(=O)c2cccc3cccc(c1=O)c32 ; *=C1C(=O)c2cccc3cccc(c23)C1=C(C#N)C#N]

and
R⁴˜R⁷ is selected from the group consisting of the following groups: hydrogen atom, halogen, cyano group, C1˜C30 linear alkyl, C3˜C30 branched alkyl, C1˜C30 silyl group, C2˜C30 ester group, C1˜C30 alkoxy, C1˜C30 thioalkyl, C1˜C30 haloalkyl, C2˜C30 alkene, C2˜C30 alkyne, C2˜C30 cyano-substituted alkyl, C1˜C30 nitro-substituted alkyl, C1˜C30 hydroxy-substituted alkyl, C3˜C30 keto-substituted alkyl.

### Example and Description of Preparation of the Organic Semiconduction Compound of the Invention

Preparation of Compound 4

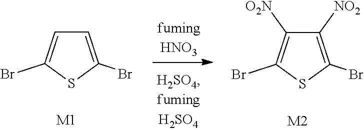

Prepare a 250 mL three-necked flask to be stirred mechanically. Pour the gas outlet of the reaction flask into NaOH_{(aq)}, and sequentially add H₂SO₄ (24.6 mL), fuming H₂SO₄ (53 mL), and fuming HNO₃ (29.2 mL) under an ice bath. Then slowly added M1 (20 g, 82.7 mmol) in portions. After adding materials, slowly bring the temperature back to room temperature and let the reaction stir for 3 hours. After the reaction, pour the reaction mixture into ice cubes and stir well. After the ice cubes are melted, collect the solid by filtration and rinse the solid with water. Recrystallize the solid with MeOH to obtain the light yellow solid M2 (24 g, at a yield of 87%). In terms of identification, since the M2 molecule does not contain hydrogen atoms, no need to do the proton NMR experiment and proceed directly to the next step of the experiment.

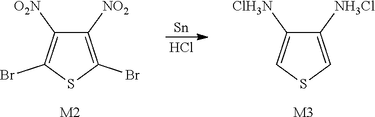

M2 (24 g, 7.23 mmol) and Conc. HCl (240 mL) was added into a 500-mL beaker and stirred with a magnet. At 0°, slowly add Sn (60 g, 50.6 mmol), and let the reaction stir for 3 hours. After the reaction, reduce the temperature of the crude product to below −20° C. and precipitate the product. Collect the cream-colored solid by filtration and rinse the solid with water to get M3 (14 g, at a yield of 60%). No need to carry out additional identification of purity, and proceed directly to the next step of the experiment.

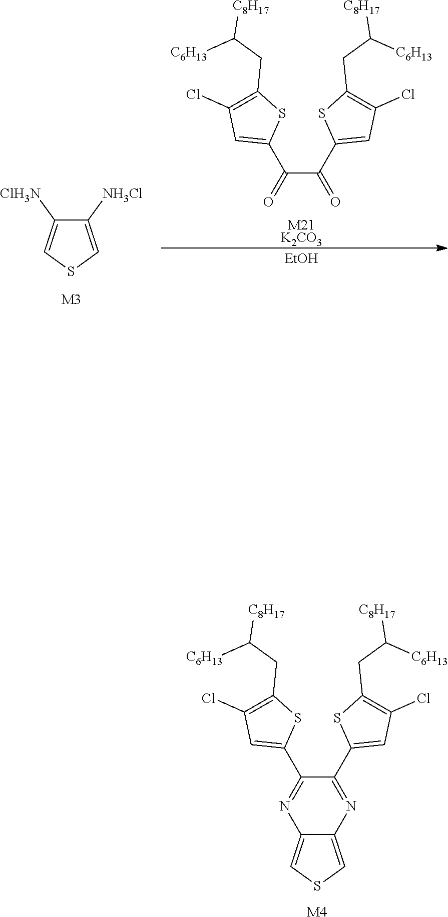

M3 (1.6 g, 8.55 mmol), M21 (7.0 g, 9.41 mmol), K₂CO₃ (2.4 g, 17.10 mmol), and EtOH (80 mL) were added into a 250 mL reaction flask and stirred with a magnet. Set the reaction temperature to 40° C., and let the reaction stir for 18 hours. After the reaction, remove the solvent and the residue was extracted three times with Heptane/H₂O, collect the organic layer, and dry with MgSO₄. Purified the crude by column chromatography (Heptane/Dichloromethane=3/1 as the eluent) to obtain the yellow-green oily material M4 (3.7 g, yield rate 53%). ¹H NMR (500 MHz, CDCl₃): δ 7.95 (s, 2H), 7.01 (s, 2H), 2.78 (d, J=7.0 Hz, 4H), 1.76 (s, 2H), 1.33-1.27 (m, 48H), 0.89-0.85 (m, 12H)

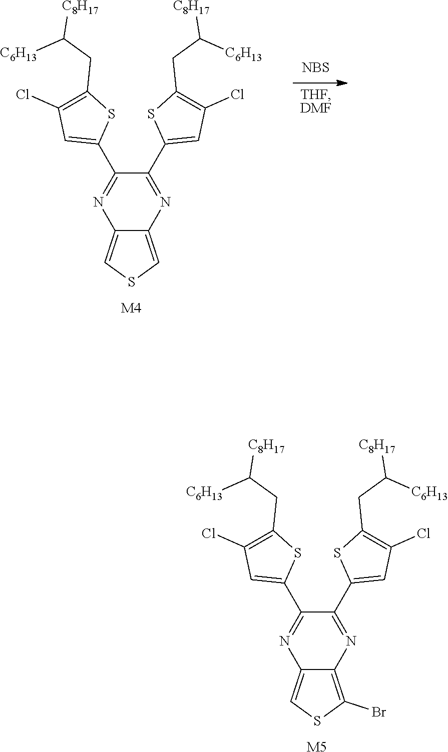

M4 (3.7 g, 4.52 mmol), THF (74 mL), and DMF (37 mL) were added into a 250 mL three-necked flask with a magnetic stir. Add NBS (724 mg, 4.07 mmol) at 0° C., and then slowly return to room temperature, and let it react for 18 hours. After the reaction, the reaction mixture was extracted three times with Heptane/H₂O, collect the organic layer, and dry with MgSO₄. Purified the crude by column chromatography (Heptane/Dichloromethane=3/1 as the eluent) to obtain the dark green oily material M5 (1.8 g, yield rate 45%). ¹H NMR (500 MHz, CDCl₃): δ 7.93 (s, 1H), 7.07 (s, 1H), 7.03 (s, 1H), 2.78-2.76 ((m, 4H), 1.76 (s, 2H), 1.33-1.25 (m, 48H), 0.89-0.85 (m, 12H)

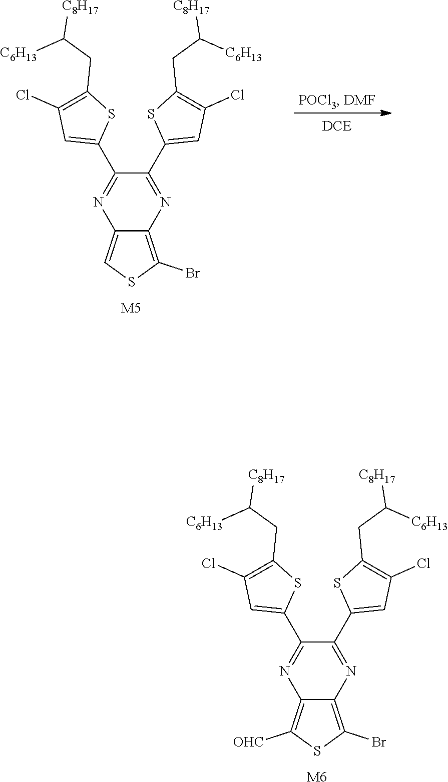

M5 (1.8 g, 2.01 mmol) and DCE (90 mL) were added into a 250 mL three-necked reaction flask with a magnetic stir, and the reaction mixture was purged with nitrogen for 30 minutes. Add anhydrous DMF (7.8 mL, 100.3 mmol) into another 100 mL, double-necked reaction flask, slowly add POCl₃ (1.1 mL, 12.0 mmol) in an ice bath, and stir for 30 minutes to produce Vilsmeier-Haack reagent. Inject the Vilsmeier-Haack reagent into the 250 mL three-necked reaction flask, and react at 65° C. for 18 hours. After the reaction, the reaction mixture was cooled to room temperature. Extracted three times with Dichloromethane/H₂O, collect the organic layer, and dry with MgSO₄. Purified the crude by column chromatography (Heptane/Dichloromethane=1/1 as the eluent) to obtain the dark green oily material M6 (1.3 g, yield rate 70%). ¹H NMR (500 MHz, CDCl₃): δ 10.57 (s, 1H), 7.21 (s, 1H), 7.18 (s, 1H), 2.81-2.78 (m, 4H), 1.78 (m, 2H), 1.34-1.28 (m, 48H), 0.89-0.86 (m, 12H)

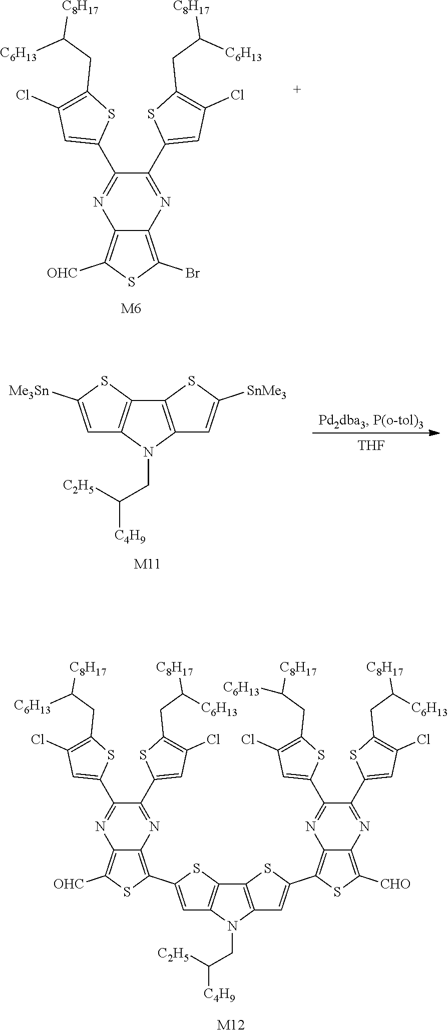

M6 (100 mg, 0.16 mmol), M11 (320 mg, 0.36 mmol), and THF (3 mL) were added into a 100 mL three-necked flask with a magnetic stir, and the reaction mixture was purged with argon for 30 minutes. Add Pd₂dba₃ (6 mg, 0.006 mmol) and P(o-tol)₃ (8 mg, 0.026 mmol) and let it react for 2 hours at 60° C. After the reaction, remove the catalyst through the Celite short column. Purified the crude by column chromatography (Heptane/Ethyl acetate=95/5 as the eluent) to obtain the dark green solid M12 (280 mg, yield rate 40%). ¹H NMR (500 MHz, CDCl₃): δ 10.54 (s, 2H), 7.91 (s, 2H), 7.25 (s, 2H), 7.24 (s, 2H), 4.18-4.13 (m, 2H), 2.86-2.80 (m, 8H), 2.05-1.93 (m, 1H), 1.84-1.82 (m, 4H), 1.48-1.23 (m, 104H), 1.01-0.88 (m, 30H)

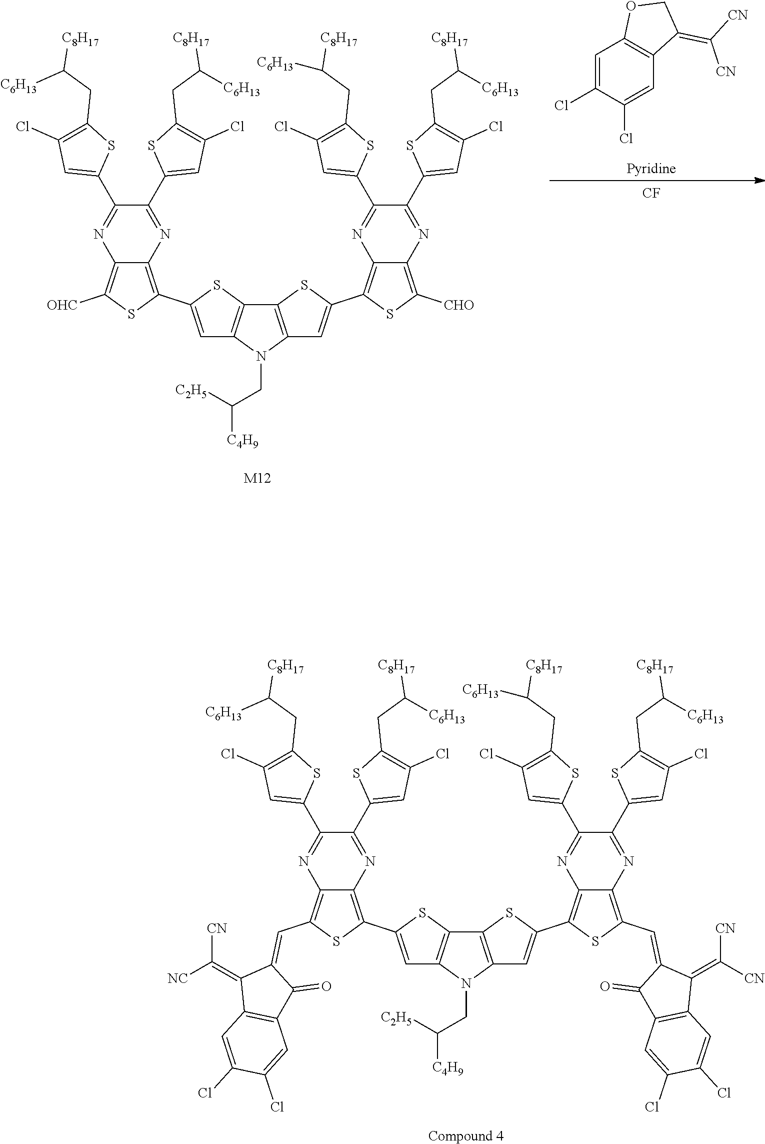

M12 (280 mg, 0.141 mmol), 2-(5,6-dichloro-3-oxo-2,3-dihydro-11-inden-1-ylidene) malononitrile (150 mg, 0.566 mmol), and CHCl₃ (8.4 mL) were added into a 100 mL three-necked flask with a magnetic stir, and the reaction mixture was purged with argon for 30 minutes. Add pyridine (0.14 mL) and let it react for 3 hours at room temperature. After the reaction, add MeOH (28 mL) and stir for 30 minutes, collect the solid by filtration. Rinse the solid with Acetone to obtain the dark blue solid Compound 4 (240 mg, yield rate 69%). ¹H NMR (500 MHz, 100° C., Cl₂CDCDCl₂): δ 9.73 (s, 2H), 8.80 (s, 2H), 7.85-7.78 (m, 4H), 7.62 (s, 2H), 7.45 (s, 2H), 4.16-4.08 (m, 2H), 3.05 (d, J=5.5 Hz, 4H), 2.68 (m, 4H), 2.13 (m, 1H), 2.10-2.00 (m, 2H), 1.77 (m, 2H), 1.58-1.36 (m, 104H), 1.14-0.92 (m, 30H)

Preparation of Compound 5

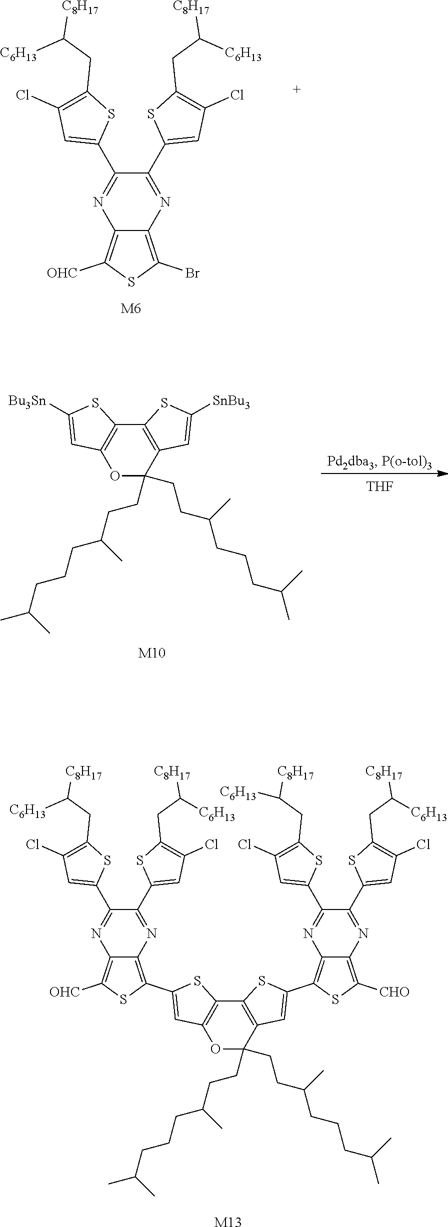

M10 (370 mg, 0.35 mmol), M6 (650 mg, 0.70 mmol), and THF (11.1 mL) were added into a 100 mL three-necked flask with a magnetic stir, and the reaction mixture was purged with argon for 30 minutes. Add Pd₂dba₃ (13 mg, 0.014 mmol) and P(o-tol)₃ (17 mg, 0.056 mmol), let it react for 2 hours at 60° C. After the reaction, remove the catalyst through the Celite short column and purified the crude by column chromatography (Heptane/Dichloromethane=1/1 as the eluent) to obtain the dark green solid M13 (500 mg, yield rate 66%). ¹H NMR (500 MHz, CDCl₃): δ 10.58 (s, 1H), 10.57 (s, 1H), 7.77 (s, 1H), 7.46 (s, 1H), 7.31-7.30 (m, 1H), 7.27 (s, 1H), 7.24 (s, 1H), 7.21 (s, 1H), 2.82 (d, J=6.5 Hz, 6H), 2.79 (d, J=7.0 Hz, 2H), 2.02-1.92 (m, 4H), 1.80 (m, 4H), 1.44-1.06 (m, 122H), 0.90-0.77 (m, 36H)

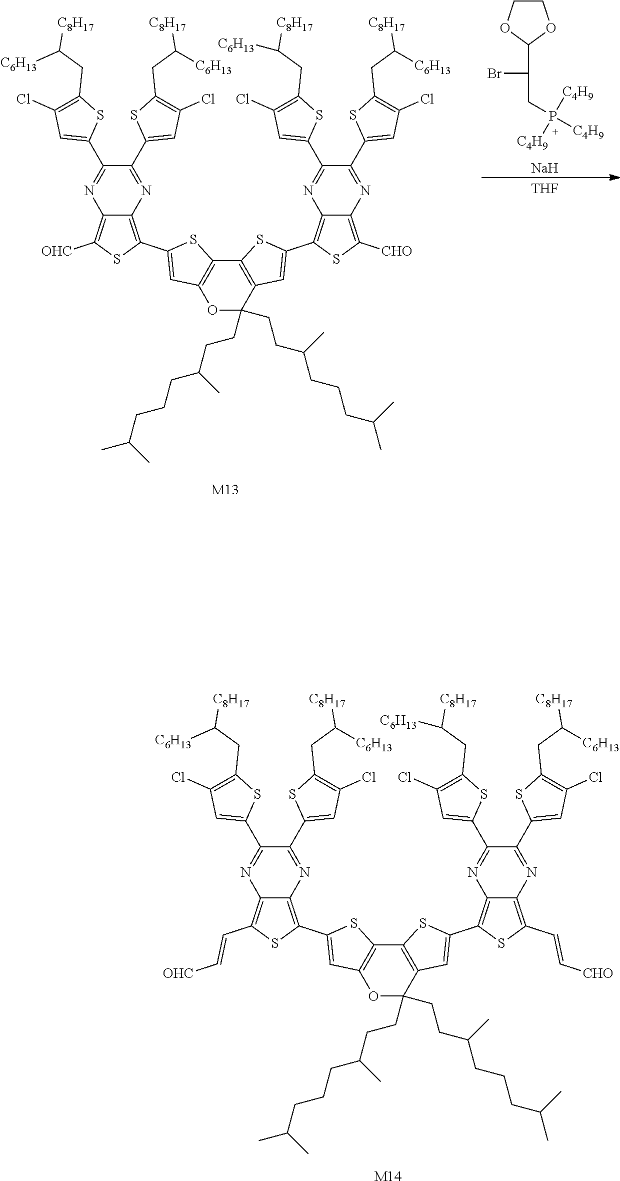

M13 (500 mg, 0.23 mmol), Tributyl (1,3-dioxolan-2-ylmethyl) phosphonium bromide (341 mg, 0.92 mmol), and anhydrous THF (15 mL) were added into a 100 mL three-necked flask with a magnetic stir. Add 60% NaH (55 mg, 1.39 mmol) at 0° C., and the reaction mixture was slowly returned to room temperature and reacted for 18 hours. Then slowly add dilute hydrochloric acid (10%, 1.5 mL) and let it react for 30 minutes at room temperature. After the reaction, extract three times with Ethyl acetate/H₂O, collect the organic layer, dry with MgSO₄, and purified the crude by column chromatography (Heptane/Ethyl acetate=9/1 as the eluent) to obtain the red solid product M14 (420 mg, yield rate 82%). ¹H NMR (500 MHz, CDCl₁): δ 9.75 (d, J=3.5 Hz, 1H), 9.74 (d, J=3.0 Hz, 1H), 8.20 (d, J=6.0 Hz, 1H), 8.17 (d, J=5.5 Hz, 1H), 7.65 (s, 1H), 7.35 (s, 1H), 7.28-7.27 (m, 1H), 7.24 (s, 1H), 7.20 (s, 1H), 7.17 (s, 1H), 6.80-6.75 (m, 2H), 2.84-2.77 (m, 8H), 2.03-1.95 (m, 4H), 1.81 (m, 4H), 1.37-1.09 (m, 122H), 0.89-0.78 (m, 36H)

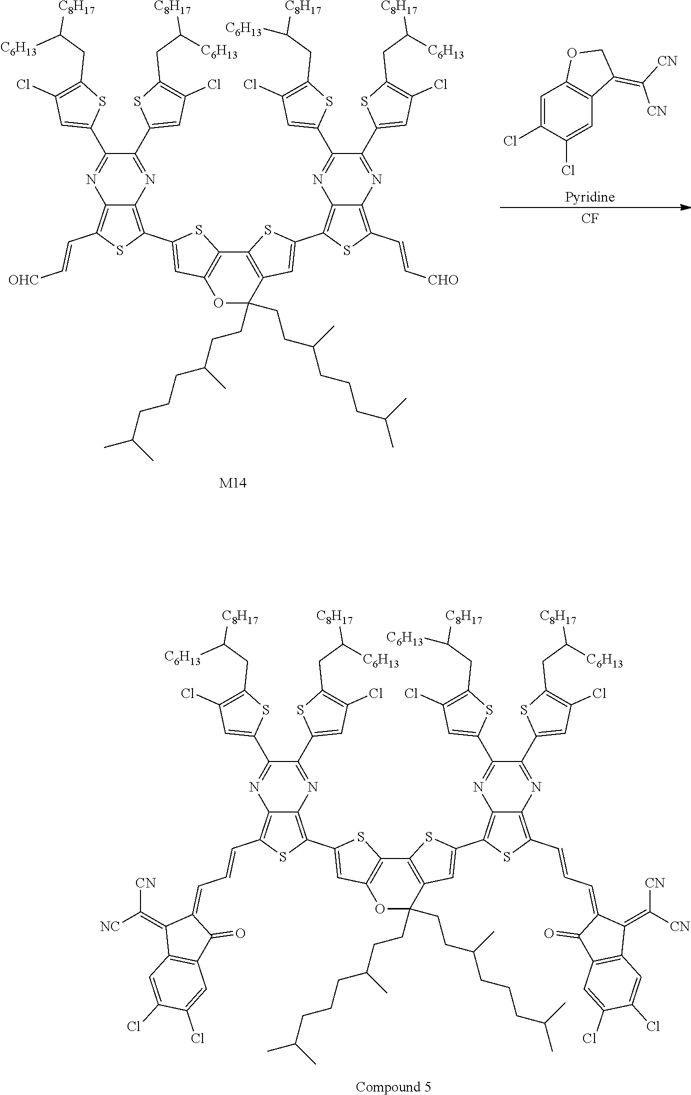

M14 (420 mg, 0.190 mmol), 2-(5,6-dichloro-3-oxo-2,3-dihydro-1H-inden-1-ylidene)malononitrile (200 mg, 0.758 mmol), and chloroform (4.2 mL) were added into a 100 mL three-necked flask with a magnetic stir, and the reaction mixture was purged with argon for 30 minutes. Add pyridine (0.21 mL) and let it react for 3 hours at room temperature. After the reaction, add in MeOH (28 mL) and stir for 30 minutes. Collect the solid by filtration. Rinse the solid with Acetone and purified the solid by column chromatography (Heptane/Chloroform=l/l as the eluent) to obtain the dark blue solid product Compound 5 (300 mg, yield rate 58%). ¹H NMR (500 MHz, 100° C., Cl₂CDCDCl₂): δ 9.09 (t, J=11.3 Hz, 1H), 9.01 (t, J=11.0 Hz, 1H), 8.78-8.77 (m, 2H), 8.55-8.53 (m, 2H), 8.13-8.05 (m, 2H), 7.96 (s, 1H), 7.95 (s, 1H), 7.90 (s, 1H), 7.58 (s, 1H), 7.51 (s, 1H), 7.50 (s, 1H), 7.37 (s, 1H), 7.34 (s, 1H), 2.94-2.90 (m, 8H), 2.18-2.10 (m, 4H), 1.94-1.90 (m, 4H), 1.62-1.17 (m, 122H), 0.98-0.87 (m, 36H)

Preparation of Compound 6

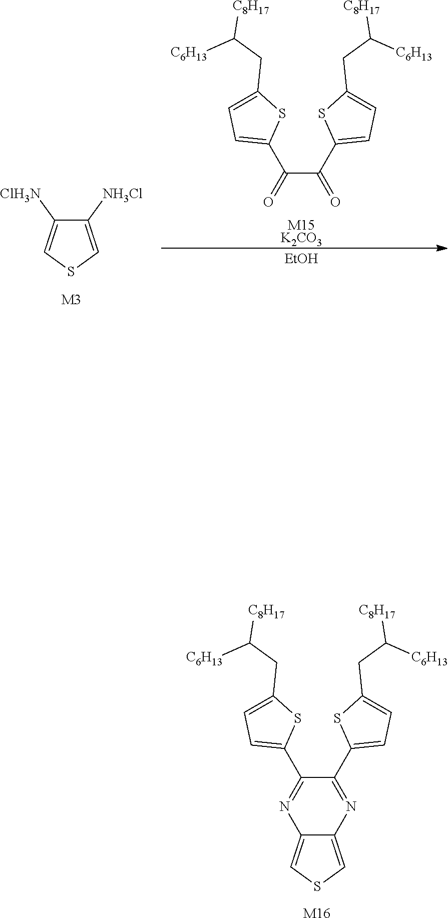

M3 (2 g, 10.69 mmol), M15 (7.9 g, 11.76 mmol), K₂CO₃ (3 g, 21.38 mmol), and EtOH (100 mL) were added into a 250 mL reaction flask with a magnetic stir, and let it react for 2 hours at 40° C. After the reaction, remove the solvent and extract it three times with Heptane/H₂O, collect the organic layer and dry with MgSO₄. Purified the crude by column chromatography (Heptane/Dichloromethane=2/1 as the eluent) to obtain the yellow-green oily material M16 (3.7 g, yield rate 46%). ¹H NMR (500 MHz, CDCl₃): δ 7.89 (s, 2H), 7.01 (d, j=4.0 Hz, 2H), 6.65 (d, J=3.5 Hz, 2H), 2.77 (d, J=7.0 Hz, 4H), 1.68 (s, 2H), 1.31-1.27 (m, 48H), 0.90-0.86 (m, 12H)

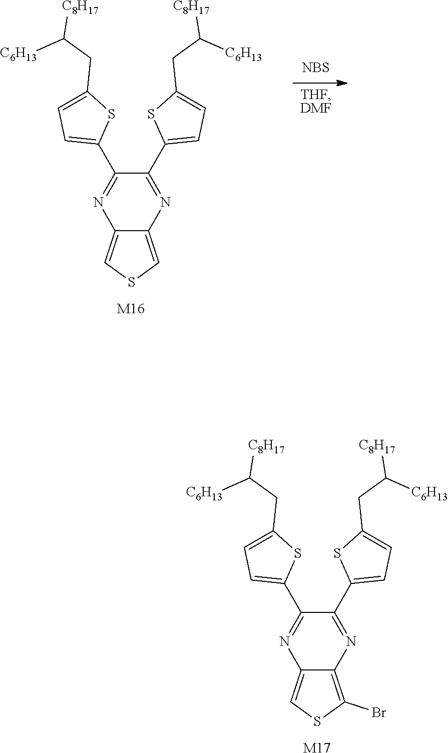

M16 (2 g, 2.67 mmol), THF (30 mL), and DMF (30 mL) were added into a 100 mL triple-necked flask. Add in NBS (475 mg, 2.67 mmol) at 0° C., the reaction mixture was slowly returned to room temperature, and reacted for 2 hours. After the reaction, extract three times with Heptane/H₂O, collect the organic layer, and dry with MgSO₄. Purified the crude by column chromatography (Heptane/Dichloromethane=3/1 as the eluent) to obtain the dark green oily material M17 (700 mg, yield rate 36%). ¹H NMR (500 MHz, CDCl₃): δ 7.90 (s, 1H), 7.10 (s, 1H), 7.07 (d, J=3.5 Hz, 1H), 6.67 (d, J=3.5 Hz, 1H), 6.64 (d, J=3.5 Hz, 1H), 2.79-2.76 (m, 4H), 1.68 (s, 1H), 1.58 (s, 1H), 1.31-1.19 (m, 48H), 0.89-0.86 (m, 12H)

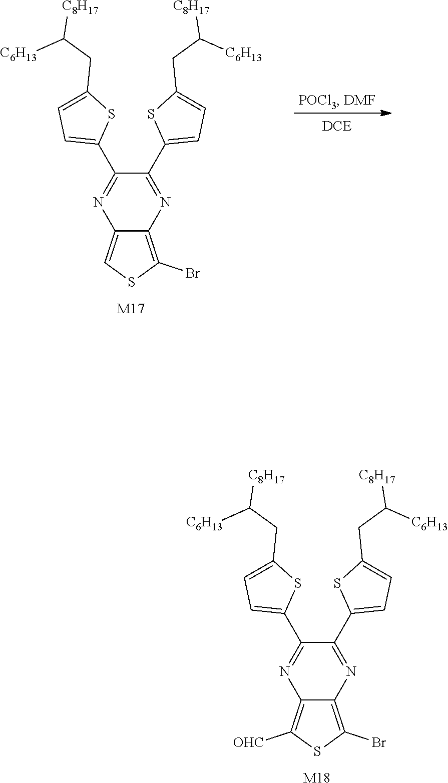

M17 (700 mg, 0.85 mmol) and DCE (35 mL) were added into a 100 mL triple-necked reaction flask with a magnetic stir, and the reaction mixture was purged with nitrogen for 30 minutes. Add anhydrous DMF (3.3 mL, 42.3 mmol) into another 100 mL double-necked reaction flask, slowly add in POCl₃ (0.5 mL, 5.07 mmol) in an ice bath, and stir for 30 minutes to produce Vilsmeier-Haack reagent. Inject the Vilsmeier-Haack reagent into the 100 mL three-necked reaction flask, and react at 65° C. for 1 hour. After the reaction, the mixture was cooled to room temperature. Extract three times with Dichloromethane/H₂O, collect the organic layer, and dry with MgSO₄. Purified the crude by column chromatography (Heptane/Dichloromethane=1/1 as the eluent) to obtain the dark green solid M18 (570 mg, yield rate 79%). ¹H NMR (500 MHz, CDCl₃): δ 10.57 (s, 1H), 7.24-7.21 (m, 2H), 6.71-6.68 (m, 2H), 2.81-2.79 (m, 4H), 1.70 (s, 2H), 1.31-1.27 (m, 48H), 0.90-0.86 (m, 12H)

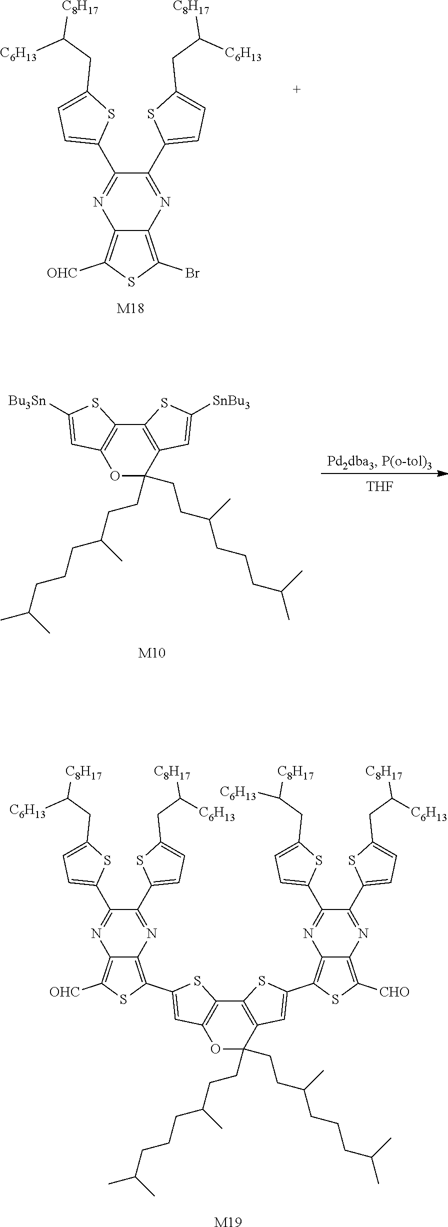

M10 (250 mg, 0.24 mmol), M18 (407 mg, 0.47 mmol), and THF (7.5 mL) were added into a 100 mL triple-necked flask with a magnetic stir, and the reaction mixture was purged with argon for 30 minutes. Add in Pd:dba₃ (9 mg, 0.009 mmol) and P(o-tol)₃ (12 mg, 0.038 mmol) and let it react for 18 hours at 60° C. Remove the catalyst through a Celite short column. Purified the crude by column chromatography (Heptane/Dichloromethane=1/1 as the eluent) to obtain the dark green solid M19 (370 mg, yield rate 72%). ¹H NMR (500 MHz, CDCl₃): δ 10.58 (s, 1H), 10.57 (s, 1H), 7.82 (s, 1H), 7.44 (s, 1H), 7.38-7.38 (m, 1H), 7.34 (d, J=4.0 Hz, 1H), 7.30 (d, J=3.5 Hz, 1H), 7.27 (s, 1H), 6.74-6.69 (m, 4H), 2.84-2.81 (m, 6H), 2.80 (d, J=7.0 Hz, 2H), 2.03-2.00 (m, 4H), 1.72 (m, 4H), 1.34-1.04 (m, 122H), 0.90-0.77 (m, 36H)

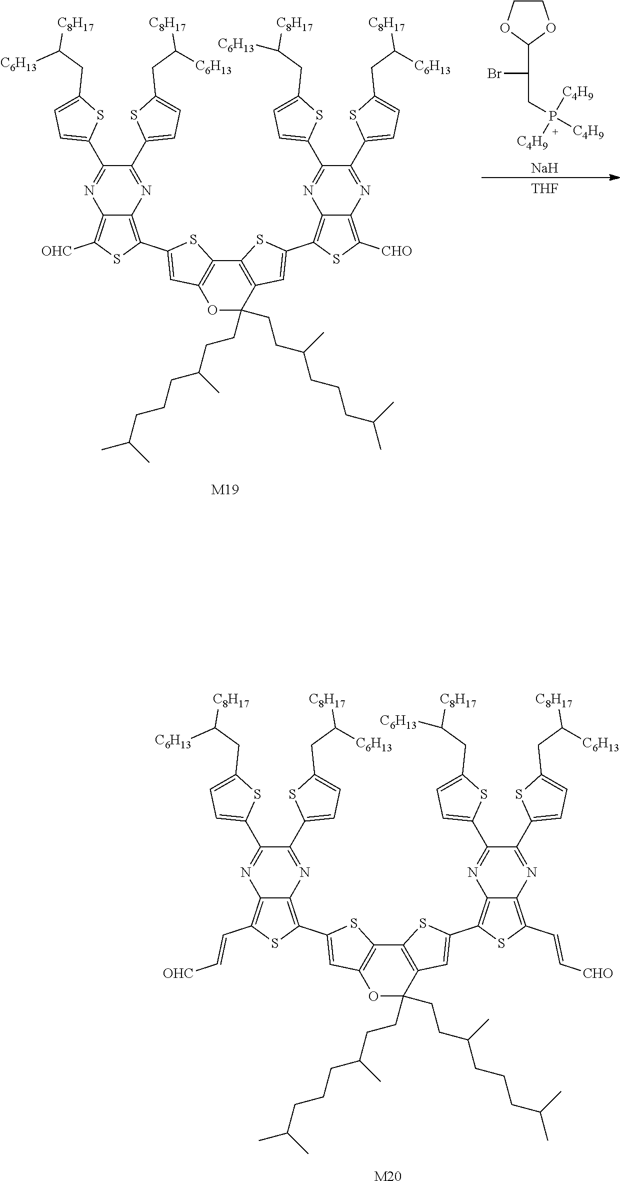

M19 (370 mg, 0.18 mmol), Tributyl (1,3-dioxolan-2-ylmethyl) phosphonium bromide (270 mg, 0.73 mmol), and anhydrous THF (11.1 mL) were added into a 100 mL triple-necked flask with a magnetic stir. Add in 60% NaH (44 mg, 1.10 mmol) at 0° C., the reaction mixture was slowly returned to room temperature, and reacted for 18 hours. Then slowly add dilute hydrochloric acid (10%, 1.11 mL) and let it react for 30 minutes at room temperature. After the reaction, extract three times with Ethyl acetate/H₂O, collect the organic layer, and dry with MgSO₄. Purified the crude by column chromatography (Heptane/Dichloromethane=1/2 as the eluent) to obtain the red solid M20 (290 mg, yield rate 76%). ¹H NMR (500 MHz, CDCl₃): δ 9.74-9.72 (m, 2H), 8.22-8.18 (m, 2H), 7.71 (s, 1H), 7.34 (s, 2H), 7.30 (d, J=4.0 Hz, 1H), 7.22 (d, J=3.5 Hz, 1H), 6.79-6.70 (m, 6H), 2.83 (d, J=6.5 Hz, 6H), 2.79 (d, J=7.0 Hz, 2H), 2.03-1.97 (m, 4H), 1.74 (m, 4H), 1.35-1.06 (m, 122H), 0.89-0.77 (m, 36H)

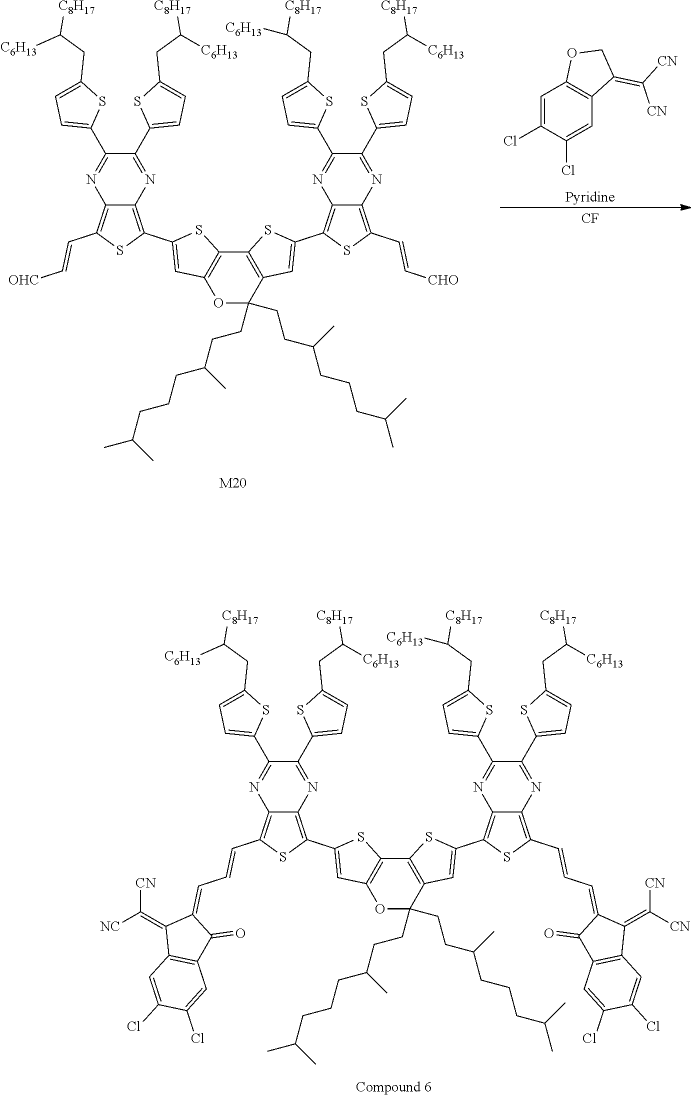

M20 (290 mg, 0.140 mmol), 2-(5,6-dichloro-3-oxo-2,3-dihydro-1H-inden-1-ylidene)malononitrile (147 mg, 0.558 mmol), and chloroform (8.7 mL) were added into a 100 mL triple-necked flask with a magnetic stir, and the reaction mixture was purged with argon for 30 minutes. Add in Pyridine (0.15 mL) and let it react for 3 hours at room temperature. After the reaction, add in MeOH (29 mL) and stir for 30 minutes, Collect the solid by filtration. Rinse the solid with Acetone to obtain the dark blue solid Compound 6 (310 mg, yield rate 88%). ¹H NMR (500 MHz, 100° C., Cl₂CDCDCl₂): δ 8.96-8.87 (m, 2H), 8.72 (s, 2H), 8.52 (m, 2H), 8.18-8.13 (m, 2H), 7.90-7.89 (m, 3H), 7.54-7.50 (m, 3H), 7.41-7.37 (m, 2H), 6.80-6.77 (m, 4H), 2.89-2.86 (m, 8H), 2.14-2.11 (m, 4H), 1.80 (m, 4H), 1.61-1.15 (m, 122H), 0.93-0.83 (m, 36H)

The example of the Organic Semiconducting Compound based on the invention is shown in Table 1

TABLE 1
Example of the Organic Semiconducting Compound of the invention

| | |
|---|---|
| 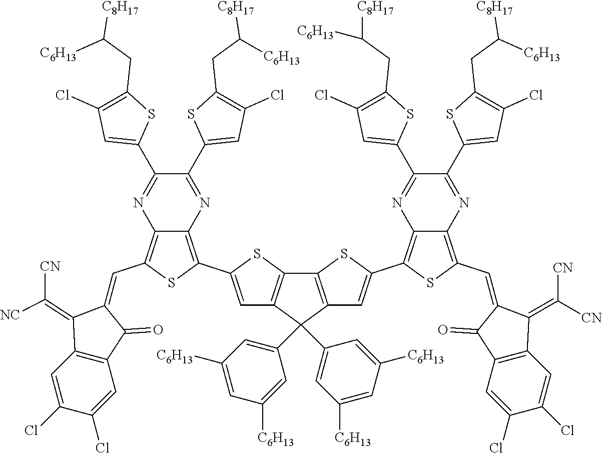 | Com- pound 1 |
| 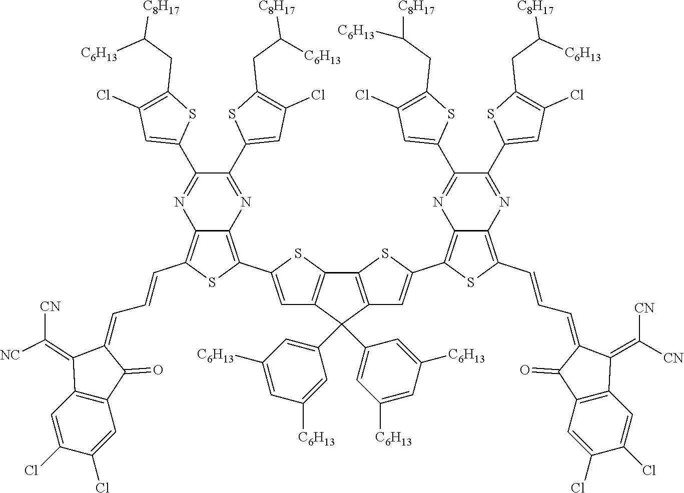 | Com- pound 2 |
| 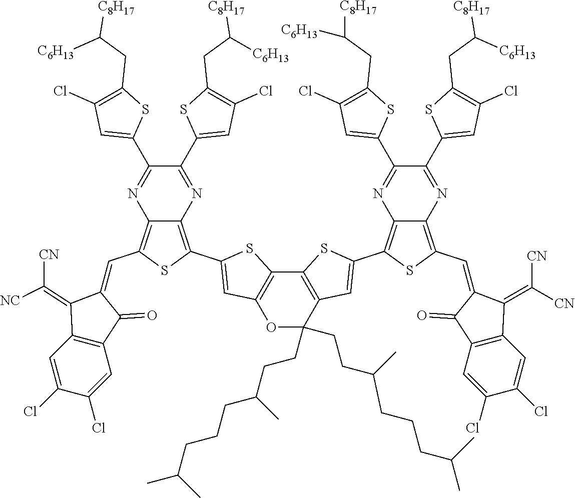 | Com- pound 3 |
| 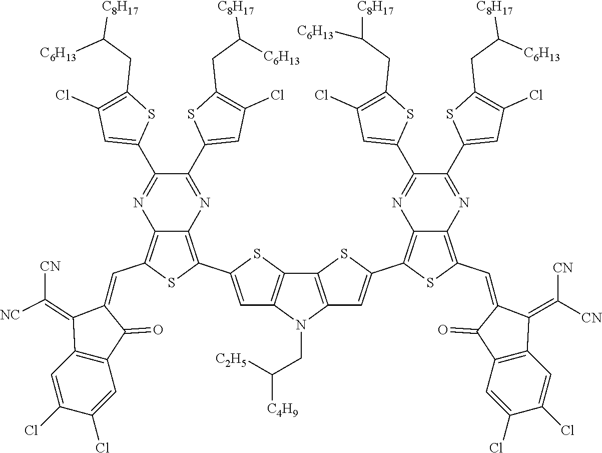 | Com- pound 4 |
| 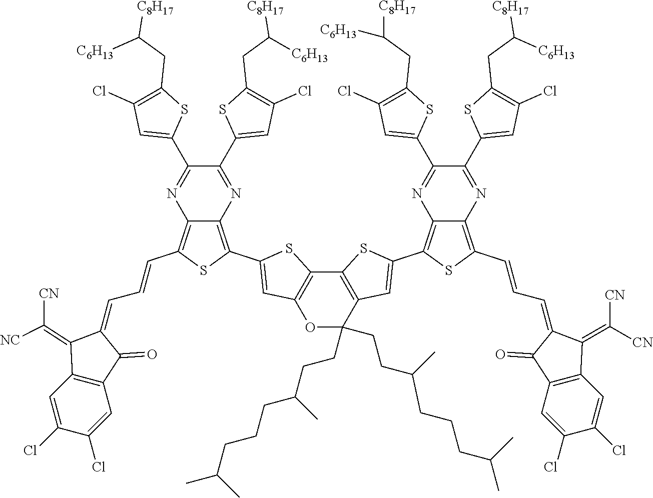 | Com- pound 5 |
| 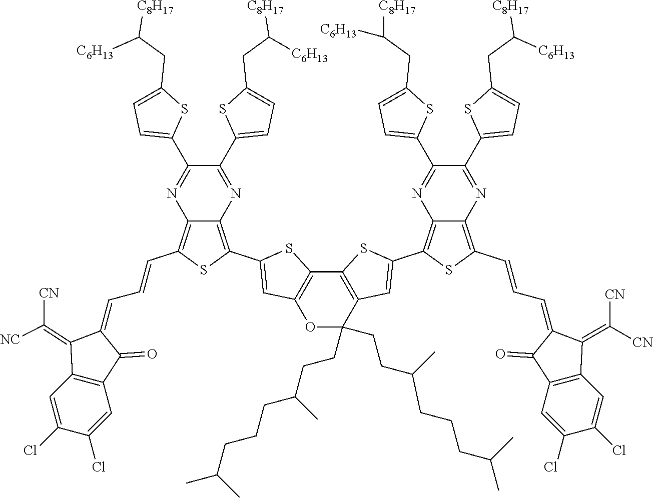 | Com- pound 6 |
| 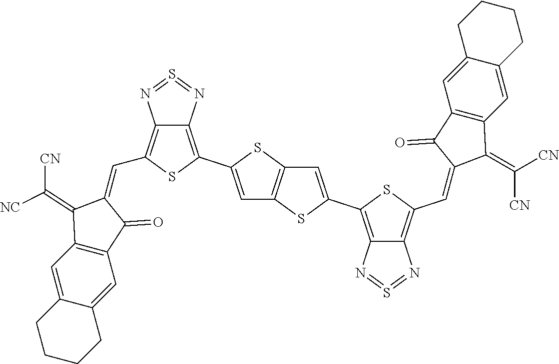 [SMILES: N#CC(C#N)=C1/C(=C/c2sc(-c3cc4sc(-c5sc(/C=C6\C(=O)c7cc8c(cc7C6=C(C#N)C#N)CCCC8)c6c5N=S=N6)cc4s3)c3c2N=S=N3)C(=O)c2cc3c(cc21)CCCC3] | Com- pound 7 |
| 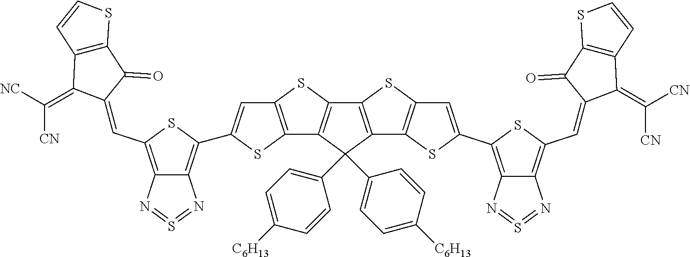 | Com- pound 8 |
| 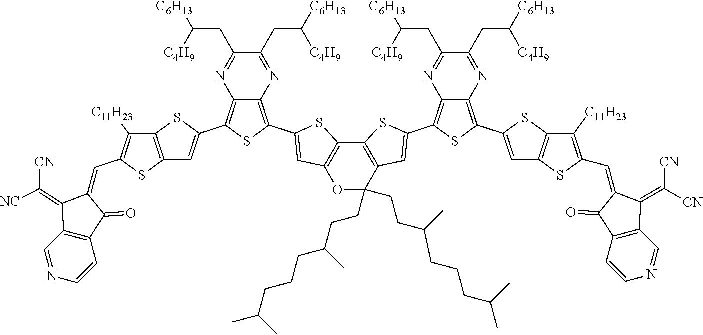 | Com- pound 9 |
| 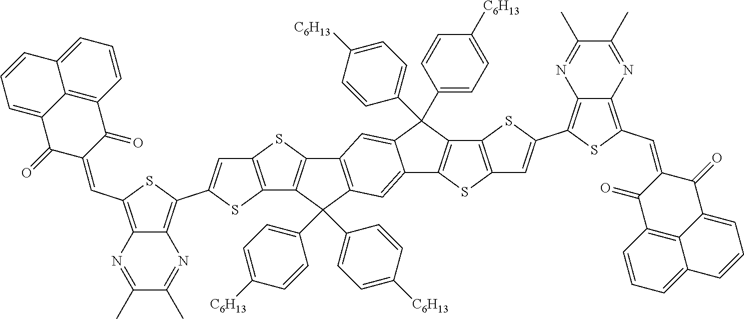 | Com- pound 10 |
| 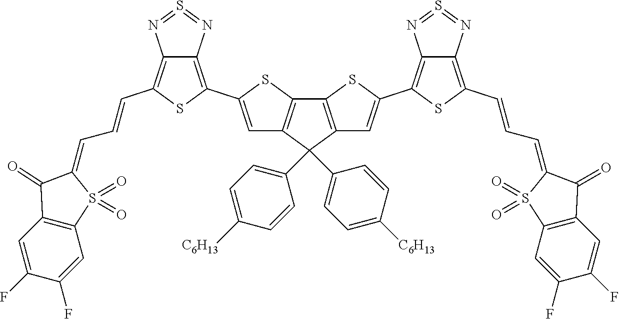 | Com- pound 11 |
| 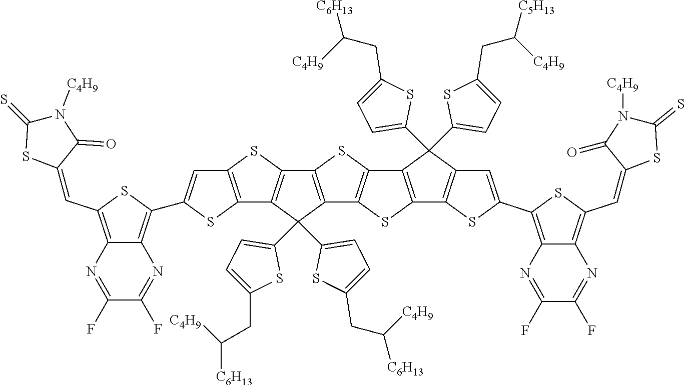 | Com- pound 12 |
| 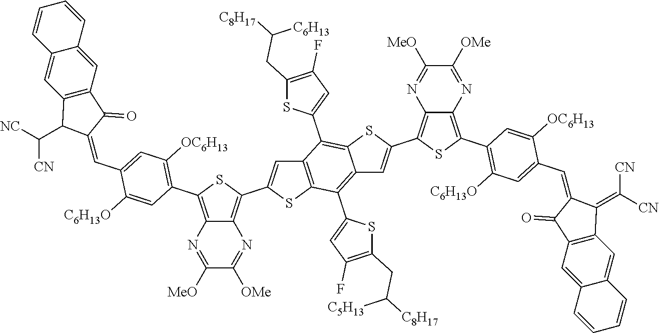 | Com- pound 13 |
| 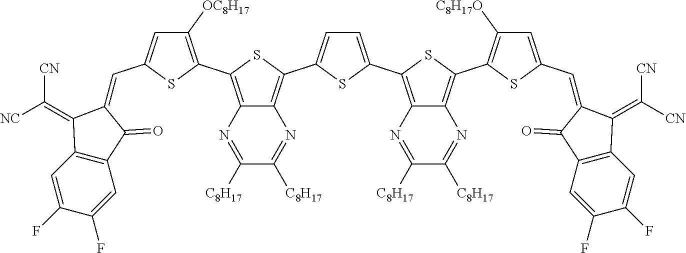 | Com- pound 14 |
| 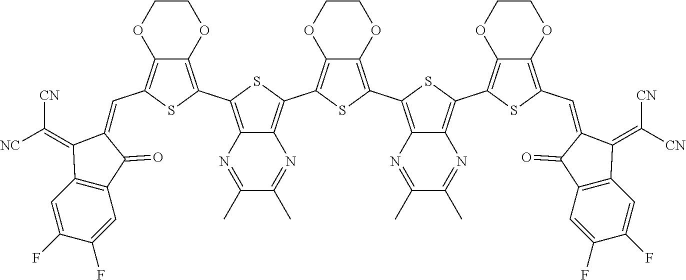 [SMILES: Cc1nc2c(-c3sc(/C=C4\C(=O)c5cc(F)c(F)cc5C4=C(C#N)C#N)c4c3OCCO4)sc(-c3sc(-c4sc(-c5sc(/C=C6\C(=O)c7cc(F)c(F)cc7C6=C(C#N)C#N)c6c5OCCO6)c5nc(C)c(C)nc45)c4c3OCCO4)c2nc1C] | Com- pound 15 |
| 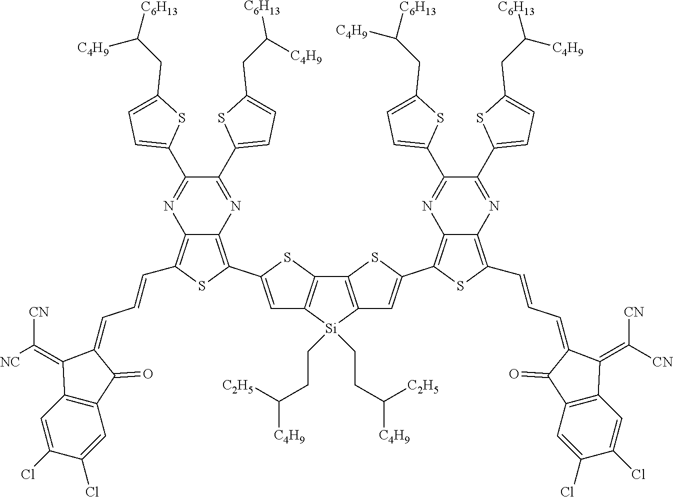 | Com- pound 16 |
| 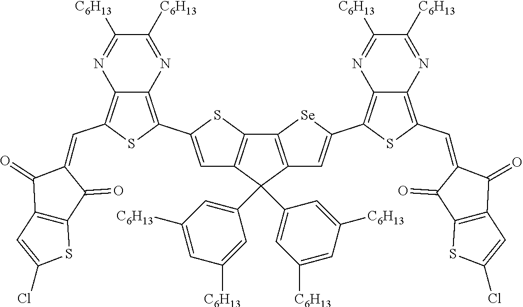 | Com- pound 17 |

Furthermore, the Organic Semiconducting Compound of the invention is used as the charge transporting, semiconducting, conductive, photoconductive, or luminescent material in an optical, electro-optical, electronic, electroluminescent or photovoltaic component or device. In such components or devices, the organic semiconducting compounds under the subject invention are generally applied as thin layers or films.

Furthermore, the organic semiconducting compounds under the subject invention are suitable to act as electron acceptors or n-type semiconductors for organic photoelectronic components, as well as suitable for preparing admixtures of n-type and p-type semiconductors for applications in fields such as organic photodetector (OPD) devices, etc. Wherein, the term “n-type” or “n-type semiconductors” refer to as extrinsic semiconductors in which the density of conducting electrons exceeds the density of mobile holes, whereas the term “p-type” or “p-type semiconductors” refers to mean Refers to extrinsic semiconductors in which the density of mobile holes exceeds the density of conducting electrons (see also J. Thewlis, Concise Dictionary of Physics, Pergamon Press, Oxford, 1973).

When the organic semiconducting compound under the subject invention is to be processed, it is necessary to introduce one or more small molecular compounds and/or polymers of the characteristics of charge transport, semi-conductivity, conductivity, photoconductivity, hole blocking, electron blocking, to be mixed therein for the preparation of the first constituent.

Furthermore, the organic semiconducting compound under the subject invention can be mixed with one or more organic solvents (preferable solvents such as aliphatic hydrocarbons, chlorinated hydrocarbons, aromatic hydrocarbons, ketones, ethers) and admixtures thereof (such as toluene, o-xylene, p-xylene, 1,3,5-trimethylbenzene or 1,2,4-trimethylbenzene, tetrahydrofuran, 2-methyltetrahydrofuran), to be mixed therein for the preparation of the second constituent.

Note that the organic semiconducting compounds under the subject invention may also be used in patterned OSC layers in devices as described herein. For microelectronic applications nowadays, it is generally desirable to produce small structures or patterns to reduce cost (i.e., more devices produced per unit area), and power consumption. The patterning of thin layers comprising the organic semiconducting compounds under the subject invention can be carried out, for example, through lithography, electron beam etching techniques, or laser patterning.

As for the application of the organic semiconducting compounds under the subject invention as the thin layers in electronic or electro-optical devices, the first constituent or the second constituent containing the organic semiconducting compounds, as prepared in the preceding paragraphs, can be deposited by any suitable methods. Solution process coating of the device is better than vacuum deposition technology. The second constituent consisting of the organic semiconducting compound under the subject invention enables the use of several solution process coating techniques.

Preferably, the deposition techniques include, but are not limited to, dip coating, spin coating, inkjet printing, nozzle printing, letterpress printing, screen printing, gravure printing, knife coating, roll printing, reverse roll printing, lithographic printing, dry offset (letterset) printing, flexographic printing, web printing, spray coating, curtain coating, brush coating, slot-dye coating, or pad printing.

Therefore, the subject invention also provides organic photoelectronic components comprising the organic semiconducting compound or the first constituent or the second constituent consisting of the organic semiconducting compound.

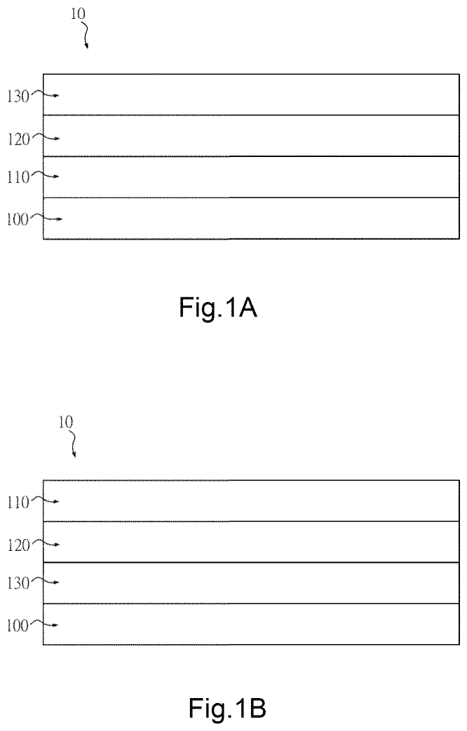

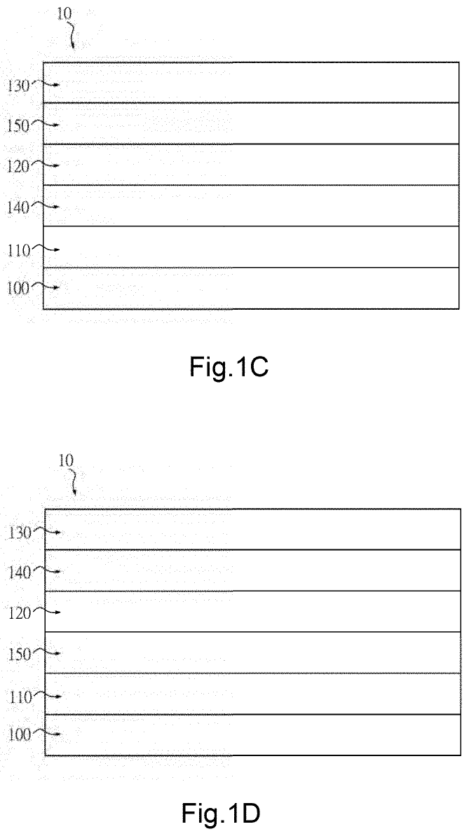

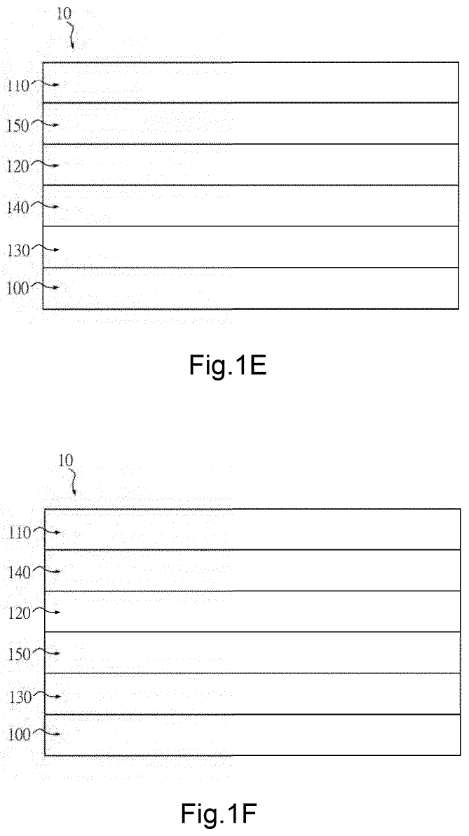

In the first embodiment of the invention as shown in FIG. 1A, the organic photoelectronic component 10 comprises: a substrate 100; a first electrode 110 which is disposed on the substrate 100; an active layer 120 which is disposed on the first electrode 110, wherein the active layer 120 includes at least one organic semiconducting compound of the present invention; and a second electrode 130 which is disposed on the active layer 120; where at least one of the first electrode 110 and the second electrode 130 is transparent or semi-transparent.

In the second embodiment of the invention as shown in FIG. 1B, the organic photoelectronic component 10 comprises: a substrate 100; a second electrode 130 which is disposed on the substrate 100; an active layer 120 which is disposed on the second electrode 130, wherein the active layer 120 comprises at least one organic semiconducting compound of the present invention; and a first electrode 110 which is disposed on the active layer 120; where at least one of the first electrode 110 and the second electrode 130 is transparent or semi-transparent.

The above-mentioned substrate 100 preferably provides a transparent glass substrate or transparent flexible substrate with mechanical strength and thermal strength, wherein the material of the transparent flexible substrate can be, namely: polyethylene, ethylene-vinyl acetate copolymer, ethylene-vinyl alcohol copolymer, polypropylene, polystyrene, polymethyl methacrylate, polyvinyl chloride, polyvinyl alcohol, polyvinyl butyraldehyde, nylon, polyetheretherketone, polysulfone, polyethersulfone, tetrafluoroethylene ethylene-pentafluoroalkyl trifluorovinyl ether copolymer, polyvinyl fluoride, tetrafluoroethylene-ethylene copolymer, tetrafluoroethylene-hexafluoropropylene copolymer, polychlorotrifluoroethylene, polyvinylidene difluoride, polyester, poly carbonate, polyurethane, polyimide, etc.

The above-mentioned first electrode 110 is preferably a metal oxide with transparency, such as indium oxide and tin oxide, etc.; as well as their derivatives doped with halogens (fluorine doped tin oxide, FTO), or composite metal oxides (e.g. indium tin oxide (ITO), indium zinc oxide (IZO), etc.

The above-mentioned second electrode 130 is a metal oxide, a metal (silver, aluminum, or gold), a conductive polymer, a carbon-based conductor, a metal compound, or a conductive film alternately composed of the above-mentioned materials.

Preferably, the active layer 120 of the organic photoelectric component 10 comprises at least one n-type Organic Semiconducting Compound which is the Organic Semiconducting Compound of the invention and at least one p-type Organic Semiconducting Compound.

More preferably, the p-type Organic Semiconducting Compound of the organic photoelectric component 10 is selected from the group consisting of the following chemical formulas:

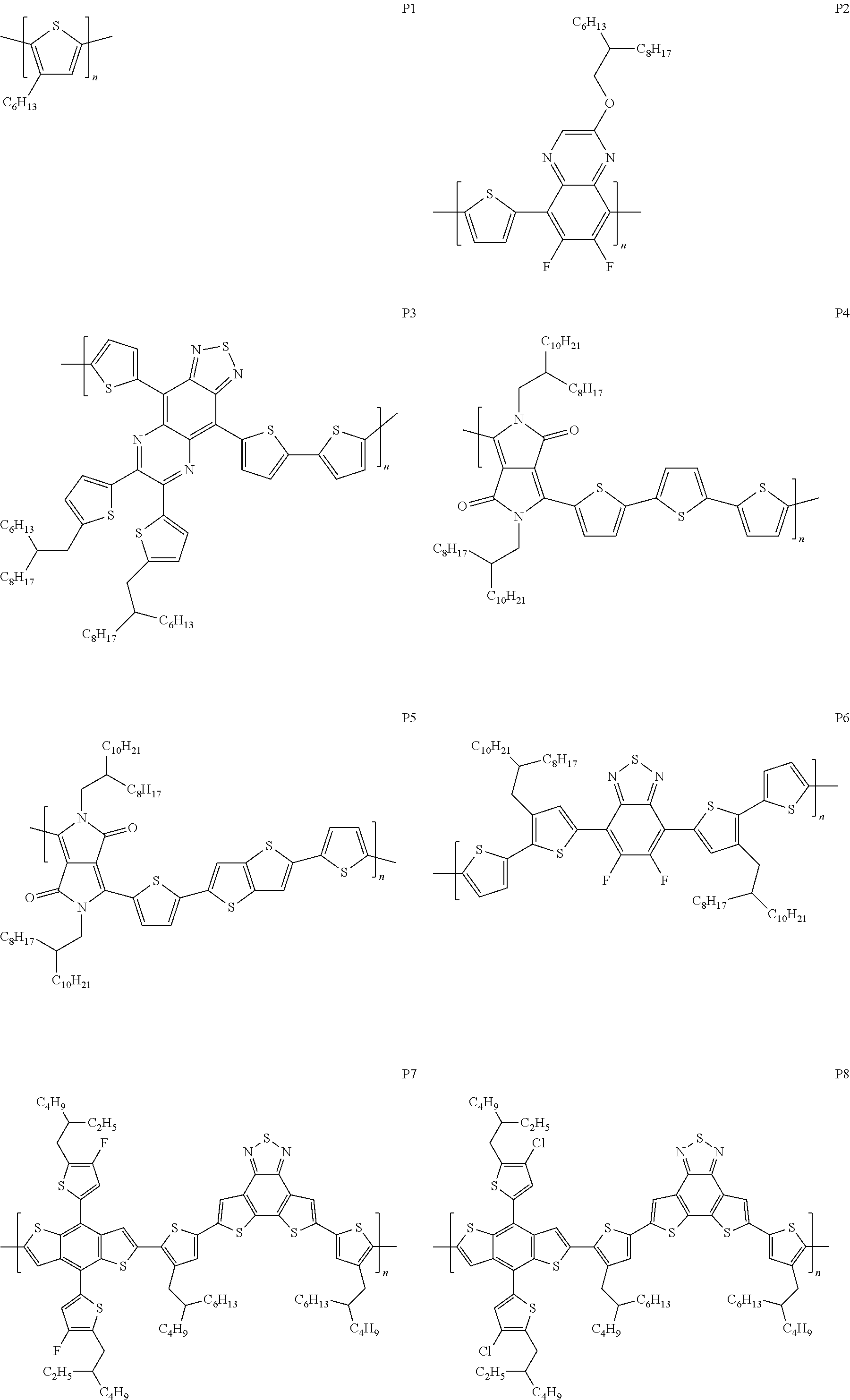

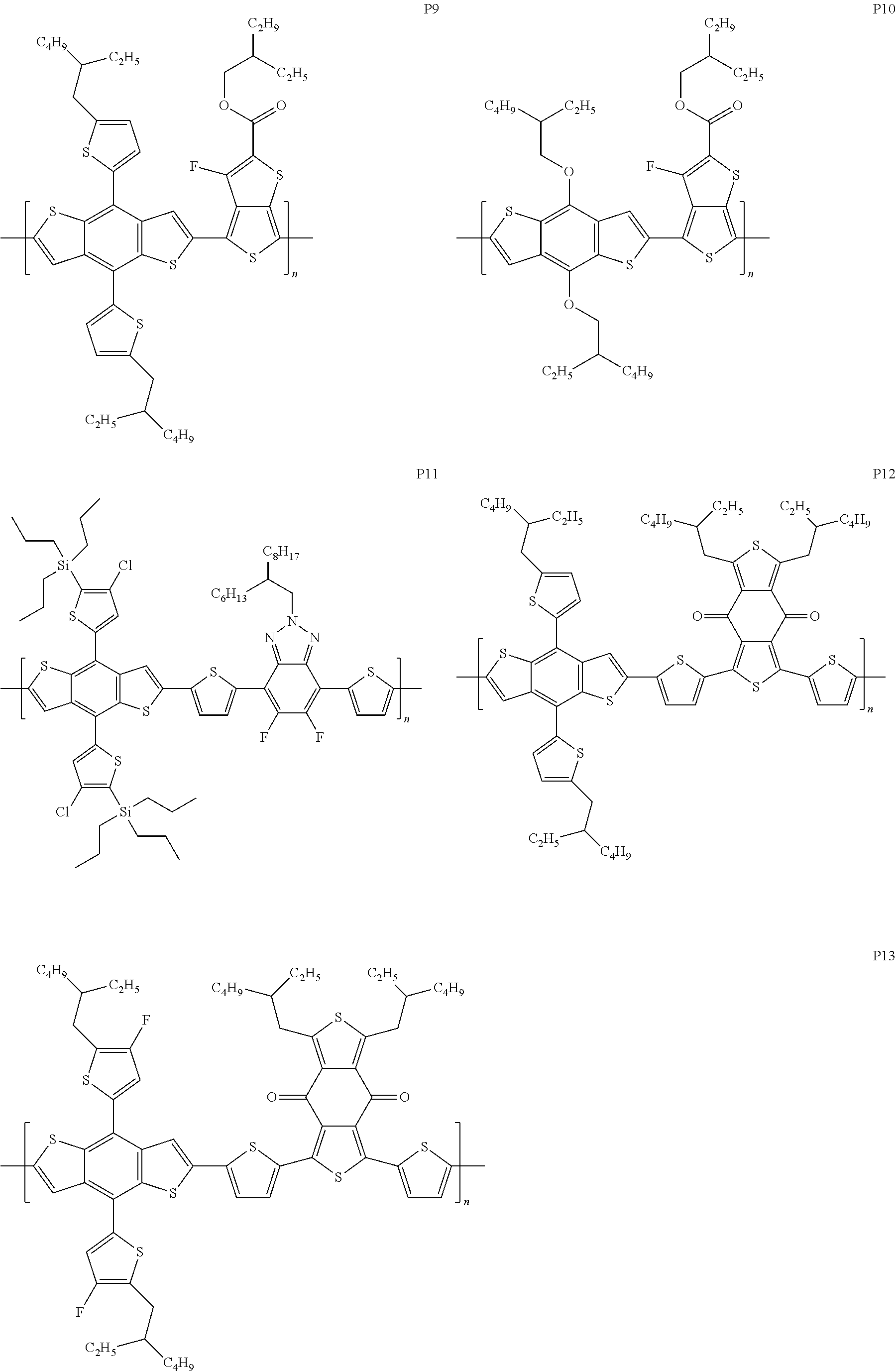

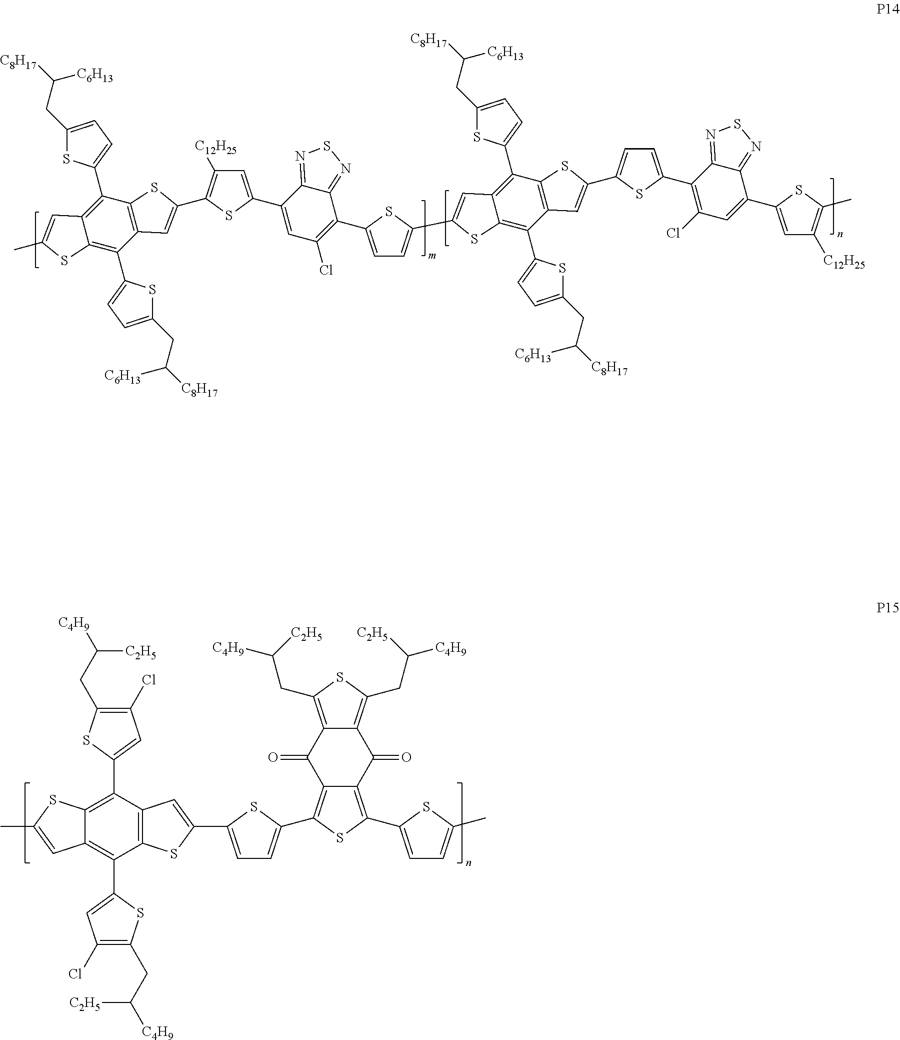

In the third embodiment of the invention as shown in FIG. 1C, the organic photoelectric component 10 further comprises: a first carrier transporting layer 140 which is disposed between the first electrode 110 and the active layer 120; and a second carrier transporting layer 150 which is disposed between the second electrode 130 and the active layer 120.

In the fourth embodiment of the invention as shown in FIG. 1D, the order of the components of the organic photoelectric component 10 is the same as in the first embodiment of the invention. The organic photoelectric component 10 further comprises: a first carrier transporting layer 140 which is disposed between the second electrode 130 and the active layer 120; and a second carrier transporting layer 150 which is disposed between the first electrode 110 and the active layer 120.

In the fifth embodiment of the invention as shown in FIG. 1E, the order of the components of the organic photoelectric component 10 is the same as in the second embodiment of the invention. The organic photoelectric component 10 further comprises: a first carrier transporting layer 140 which is disposed between the second electrode 130 and the active layer 120; and a second carrier transporting layer 150 which is disposed between the first electrode 110 and the active layer 120.

In the sixth embodiment of the invention as shown in FIG. 1F, the order of the components of the organic photoelectric component 10 is the same as in the second embodiment of the invention. The organic photoelectric component 10 further comprises: a first carrier transporting layer 140 which is disposed between the first electrode 110 and the active layer 120; and a second carrier transporting layer 150 which is disposed between the second electrode 130 and the active layer 120.

In the aforementioned third to sixth implementation approach, the first carrier transporting layer may be selected from conjugated polymer electrolytes, such as PEDOT:PSS; or polymer acids, such as polyacrylates; or conjugated polymers, such as polytriarylamine (PTAA); or insulating polymers, such as Nafion films, polyethyleneimine, or polystyrene sulfonate; or polymer-doped metal oxides, such as MoOx, NiOx, WOx, SnOx; or organic small molecule compounds, such as N,N′-diphenyl-N,N′-bis(t-naphthyl)(1,1′-biphenyl)-4,4′-diamine (NPB), N,N′-diphenyl-N,N′-(3-methylphenyl)-1,1′-biphenyl-4,4′-diamine (TPD); or a combination of one or more of the above-mentioned materials.

In the aforementioned third to sixth implementation approach, the second carrier transporting layer can be selected from conjugated polymer electrolytes, such as polyethyleneimine; or conjugated polymers, such as poly[3-(6-trimethyl) ammoniumhexyl)thiophene], poly(9,9)-bis(2-ethylhexyl-fluorene)-b-poly[3-(6-trimethylammoniumhexyl)thiophene], and poly[(9,9-bis(3′-(N,N-dimethylamino)propyl)-2,7-fluorene)-alt-2,7-(9,9-dioctylfluorene)]; or small organic compounds, such as tris (8-quinolinyl)-aluminum (Ill)(Alq3), 4,7-diphenyl-1,10-phenanthroline; or metal oxides, such as ZnOx, aluminum-doped ZnO (AZO), TiOx or its nanoparticles; or salts such as LiF, NaF, CsF, CsCO3; or amines such as primary, secondary, or tertiary amines.

To illustrate the efficacy improvement brought by the organic semiconducting compound under the subject invention applied to an organic photoelectronic component, the organic photoelectronic components comprising the organic semiconducting compound under the subject invention will be prepared for property testing and efficacy performance. The test results are as follows:

Material Absorbance Spectral Test

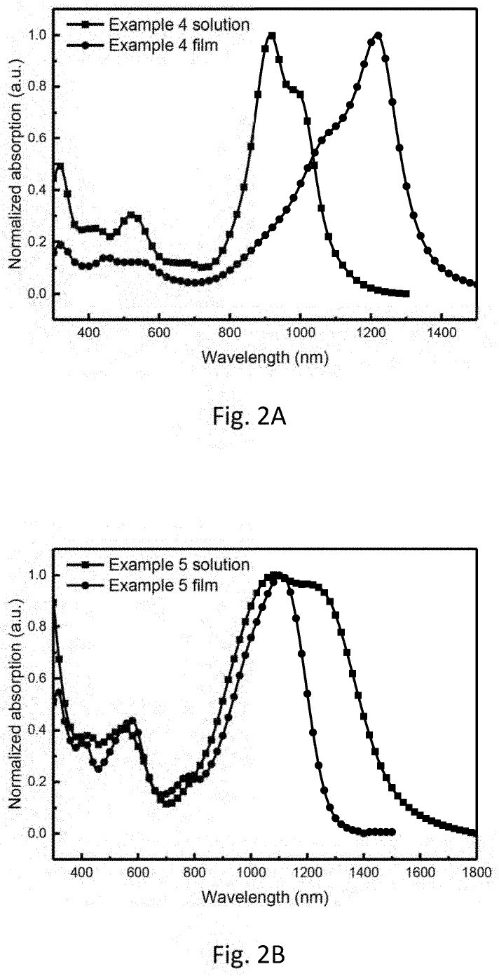

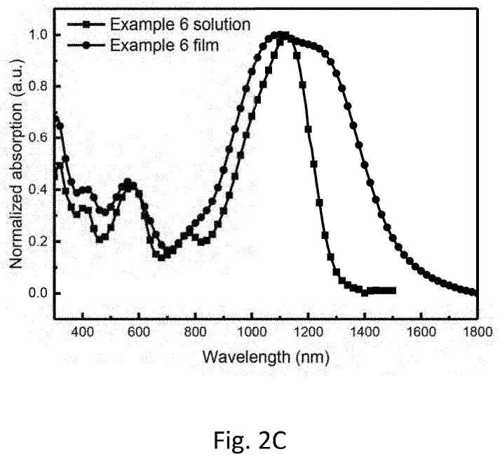

Use a UV-visible spectrometer to detect the absorption spectrum of the samples. First, apply chloroform to dissolve the samples before the measurement of samples. When the solid state is measured, the samples must be prepared into a thin film before the measurement can be taken. To preparation of thin film samples: Configure the concentration of the sample to be 5 wt %. Use the glass as the substrate and coat the thin film on the glass by spin coating, then measure the absorption of the solid thin film. The absorption spectra of each sample are shown in FIG. 2A to FIG. 2C, and the measurement results are shown in Table 2.

TABLE 2
Absorbance spectrum of the sample and results of the electrochemical property test

| | λₛₒₗₙ^{max} | λ_{film}^{max} | λ_{film}^{onset} | ε | E_{g}^{opt} | HOMO | LUMO |
|---|---|---|---|---|---|---|---|
| Material | (nm) | (nm) | (nm) | (10⁵ cm⁻¹M⁻¹) | (eV) | (eV) | (eV) |
| Compound 4 | 917 | 1217 | 1339 | 1.14 | 0.93 | −5.48 | −4.55 |
| Compound 5 | 1104 | 1105, 1219 | 1510 | 0.95 | 0.82 | −5.41 | −4.59 |
| Compound 6 | 1123 | 1096, 1237 | 1529 | 0.89 | 0.81 | −5.31 | −4.50 |
| Comparative | 700 | 780 | 847 | 1.3 | 1.47 | −5.51 | −4.02 |
| Example 1 | | | | | | | |
| Comparative | 770 | 835 | 1333 | — | 0.93 | −4.62 | −3.69 |
| Example 2 | | | | | | | |
| Comparative | 714 | 865 | 1220 | — | 0.98 | −4.71 | −3.59 |
| Example 3 | | | | | | | |

The results of tests of the organic photoelectric components prepared with Compounds 4, 5, and 6 showed that all three materials have good solubility (14 mg/mL, in o-xylene). Compound 4 has an absorbance onset value of 1339 nm, while Compound 5 and 6 even have an absorbance onset value of 1510 and 1529 nm, indicating that the Organic Semiconducting Compound of the invention has excellent absorbance properties above 1000 nm and is more suitable for longer wavelength range applications. Comparative Example 1 is from J. Mater. Chem. C, 2019, 7, 8820-8824; Comparative Example 2 is from Polym. Chem. 2015, 6, 6836-6844: Comparative Example 3 is the compound revealed in Appl. Phys. Lett. 2006, 89, 081106, the above documents are the source referred to for the control group of the invention. The absorbance onset value of Comparative Example 1 and Comparative Example 3 was only 1200 nm. Though the absorbance onset value of Comparative Example 2 reaches 1333 nm, the maximum value is only 835 nm, which means its absorbance spectrum is not sufficient above 1000 nm. Therefore, apart from the innovative structure of the organic semiconductor material of the invention, it is invented with advanced technology that enables the extension of the absorbance spectrum to cover the long infrared section.

Electrochemical Properties Test

Use an electrochemical analyzer to record the oxidation and reduction potentials. Take 0.1 M Bu₄NPF₆ (tetra-1-butylammonium hexafluorophosphate) acetonitrile solution as the electrolyte. 0.01 M AgNO₃ (silver nitrate) and 0.1 M TBAP (tetrabutylammonium perchlorate) in acetonitrile solution was added into the Ag/AgCl reference electrode. Take platinum (Pt) as the counter electrode, and carbon glass electrode as the working electrode. Using chloroform to dissolve the materials and drop them onto the working electrode, after drying, a thin film was formed. During the measurement, adopt the scan rate of 50 mV/sec and record the redox curve at the same time. When making a CV figure, its redox potential can be obtained. Take ferrocene/ferrocenium (Fc/Fc⁺) as an internal reference potential, and with the correction, and derive the HOMO and LUMO values. The calculation formula is as follows:

HOMO=−(4.71 eV+(Eₒₓ−E_{ref}))

LUMO=HOMO+E_{g}^{opt}

The results of test of each sample are shown in Table 2.

OPD Performance Test

Take a pre-patterned ITO-coated glass with sheet resistivity as the substrate. Sonicate sequentially the substrate in a neutral detergent, deionized water, acetone, and isopropanol. In each of such step, carry out the rinsing for 15 minutes. The washed substrate was further treated with a UV-O₃ cleaner for 15 minutes. Apply the top coating of AZO (aluminum doped zinc oxide nanoparticle) on the ITO substrate at a spin rate of 2,000 rpm for 40 seconds. Dry the substrate with heat at 120° C. in the air for 5 minutes. Prepare the active layer solution in o-xylene (where the weight ratio of the donor, i.e., polymer, to the acceptor, i.e., small molecule, is 1:1). The concentration of the polymer is 20 mg/ml. To completely dissolve the polymer, the active layer solution should be stirred on a hot plate at 100° C. for at least 3 hours. Filter the active layer solution through a PTFE membrane filter (with pore size 0.45˜1.2 μm), and heat the active layer solution for 1 hour. Then, let the solution cool down to room temperature before coating. Control the coating speed so that film thickness is within the range of 100˜300 nm. Once done, the thin films were annealed at 100° C. for 5 minutes and then transfer the thin films to an evaporator. Deposit of a thin layer (8 nm) of molybdenum trioxide as a hole transporting layer under 3×10⁻⁶ torr. Use a Keithley™ 2,400 source meter to record the dark current (ID, bias voltage at −8V) in the absence of light. Next, use a solar simulator (a xenon lamp with an AM 1.5G filter, 100 mW cm²) to measure photocurrent (Iv) characteristics of the component in air and at room temperature. Use a standard silicon diode with a KG5 filter is used as a reference cell for the calibration of the light intensity so that the mismatched portion on the spectrum can reach consistency. Use an external quantum efficiency meter for the measurement of the external quantum efficiency (EQE), with a measurement range of 300˜1,800 nm (bias voltage of 0˜−8V). Use silicon (300˜1,100 nm) and germanium (1,100˜1,800 nm) for calibration of the light source. In addition, the responsibility (R) and detectivity (D) are calculated by the following formulas:

$$R ( λ ) = EQE \frac{λ q}{hc}$$

$$D = \frac{R}{\sqrt{2 {qJ}_{D}}}$$

where λ is the wavelength, q is the unit charge, h is Planck constant, c is the speed of light, and J_{D} is the dark current density.

Comparative Example 3 of the invention is quoted from the experimental results of Appl. Phys. Lett. 2006, 89, 081106. Since the test values of responsivity and detectivity are not directly listed in Comparative Example 3, the values in Table 3 are calculated based on the experimental data of this article.

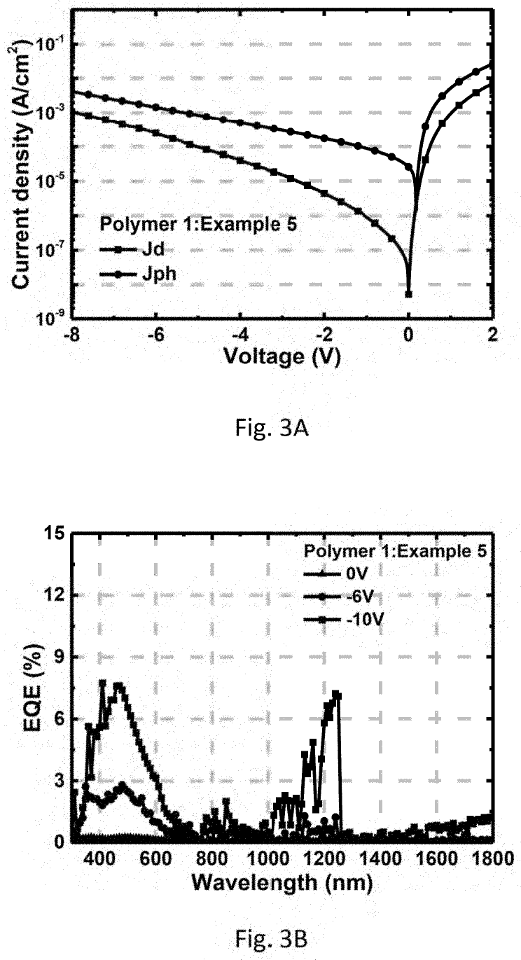

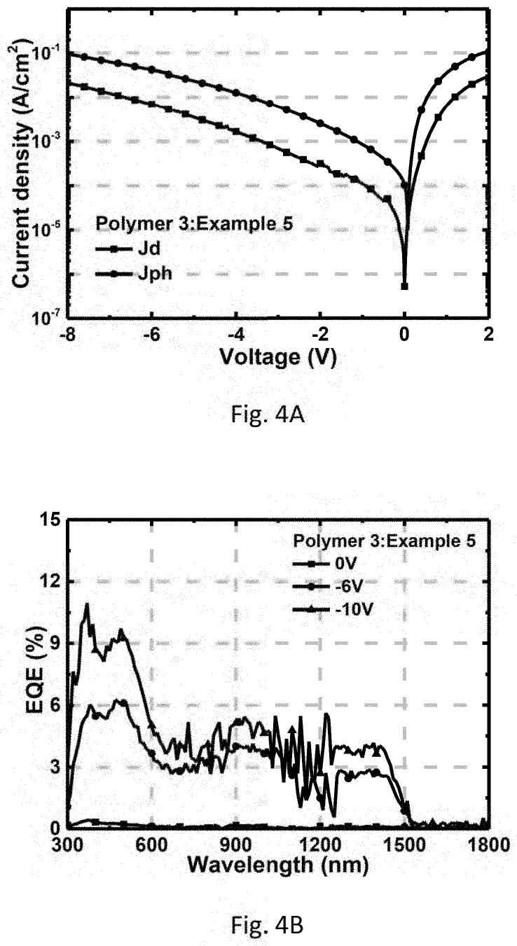

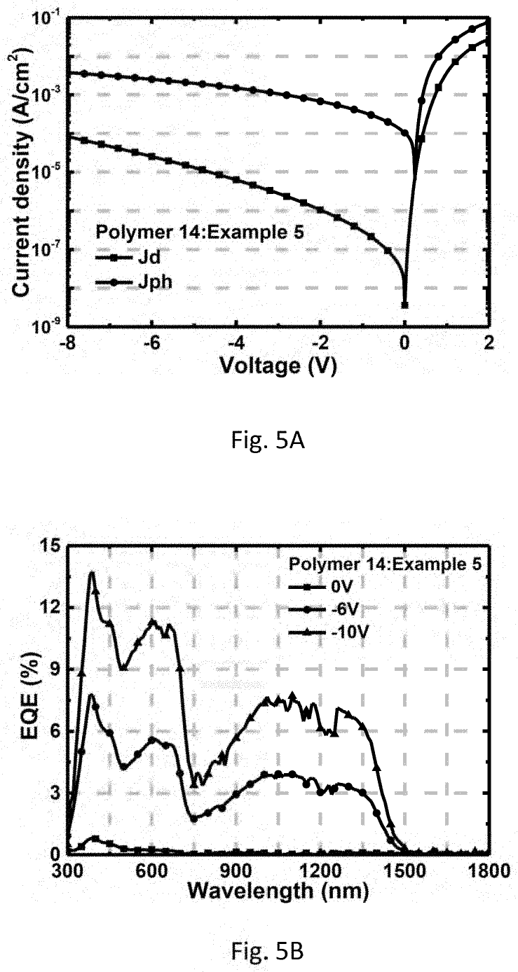

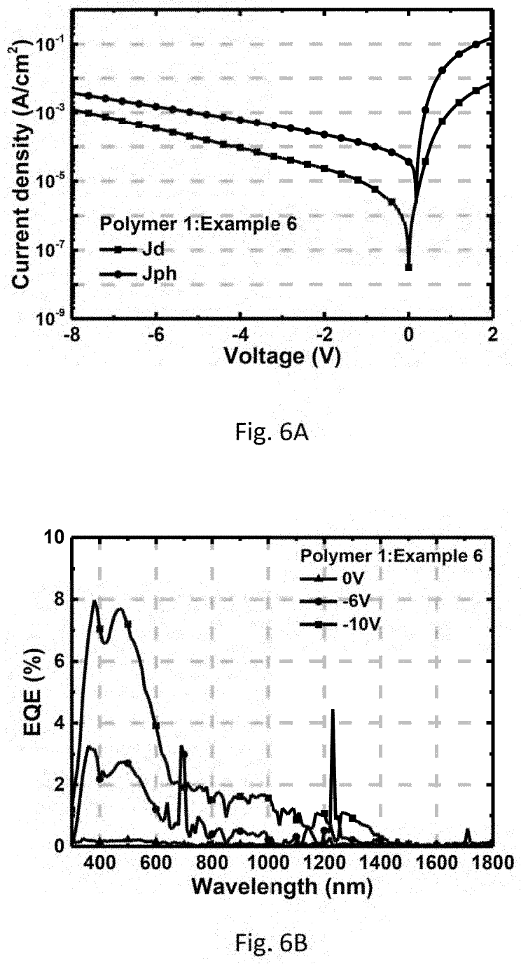

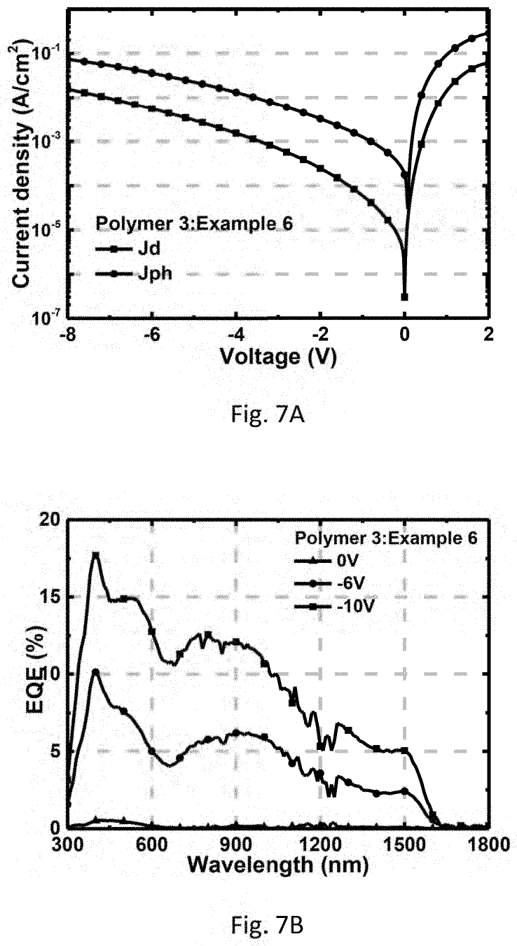

The current density and external quantum efficiency of each sample are shown in FIGS. 3A, 3, 4A 4, 5A, 5B, 6A, 6B, 7A and 7B, and the results of tests are shown in Table 3 and Table 4.

TABLE 3
Electrical property test of the organic photoelectric component
containing the Organic Semiconducting Compound of the invention

| | | J_{dark} | J_{dark} | R¹⁰⁵⁰ | D¹⁰⁵⁰ | R¹⁰⁵⁰ | D¹⁰⁵⁰ |
|---|---|---|---|---|---|---|---|
| ATL 100 nm | | at −2 V | at −8 V | at −2 V | at −2 V | at −8 V | at −8 V |
| Donor/Acceptor | | (A/cm²) | (A/cm²) | (A/W) | (Jones) | (A/W) | (Jones) |
| P1 | Compound 5 | 4.4 × 10⁻⁶ | 1.1 × 10⁻³ | 1.3 × 10⁻³ | 1.1 × 10⁹ | 7.8 × 10⁻³ | 4.1 × 10⁸ |
| P3 | | 3.1 × 10⁻⁴ | 2.3 × 10⁻² | 1.1 × 10⁻² | 1.1 × 10⁹ | 3.3 × 10⁻² | 4.7 × 10⁸ |
| P14 | | 1.1 × 10⁻⁶ | 8.2 × 10⁻⁵ | 7.6 × 10⁻³ | 1.3 × 10¹⁰ | 4.7 × 10⁻² | 9.3 × 10⁹ |
| P1 | Compound 6 | 2.3 × 10⁻⁵ | 1.3 × 10⁻³ | 4.2 × 10⁻⁴ | 1.6 × 10⁸ | 6.3 × 10⁻³ | 3.1 × 10⁸ |
| P3 | | 2.4 × 10⁻³ | 1.6 × 10⁻² | 1.3 × 10⁻² | 4.6 × 10⁸ | 6.3 × 10⁻² | 8.8 × 10⁸ |
| Comparative | PCBM | ~10⁻³ | — | 2 × 10⁻³ | 6.5 × 10⁷ | — | — |
| Example 3 | | | | | | | |

TABLE 4
Electrical property test of the organic photoelectric component
containing the Organic Semiconducting Compound of the invention

| | | J_{dark} | J_{dark} | R¹³⁵⁰ | D¹³⁵⁰ | R¹³⁵⁰ | D¹³⁵⁰ |
|---|---|---|---|---|---|---|---|
| ATL 100 nm | | at −2 V | at −8 V | at −2 V | at −2 V | at −8 V | at −8 V |
| Donor/Acceptor | | (A/cm²) | (A/cm²) | (A/W) | (Jones) | (A/W) | (Jones) |
| P1 | Compound 5 | 4.4 × 10⁻⁶ | 1.1 × 10⁻³ | 8.7 × 10⁻⁴ | 7.3 × 10⁸ | 1.5 × 10⁻³ | 8.1 × 10⁷ |
| P3 | | 3.1 × 10⁻⁴ | 2.3 × 10⁻² | 1.1 × 10⁻² | 1.1 × 10⁹ | 3.9 × 10⁻² | 5.5 × 10⁸ |
| P14 | | 1.1 × 10⁻⁶ | 8.2 × 10⁻⁵ | 7.6 × 10⁻³ | 1.3 × 10¹⁰ | 4.8 × 10⁻² | 9.4 × 10⁹ |
| P1 | Compound 6 | 2.3 × 10⁻⁵ | 1.3 × 10⁻³ | 9.8 × 10⁻⁴ | 3.6 × 10⁸ | 7.4 × 10⁻³ | 3.6 × 10⁸ |
| P3 | | 2.4 × 10⁻³ | 1.6 × 10⁻² | 7.6 × 10⁻³ | 2.7 × 10⁸ | 6.1 × 10⁻² | 8.6 × 10⁸ |

For EQE performance, both Compound 5 and Compound 6 exceeded 1000 nm, and the dark current reached 1.1×10⁻⁶ and 2.3×10⁻⁵ A/cm² respectively at a bias of −2 V. The responsivity of P3 with Compound 6 at 1050 nm was 0.013 A/W, and the responsivity of P3 with Compound 5 at 1350 nm was 0.011 A/W. The results were significantly improved compared with Comparative Example 3 (<0.01 A/W). For detectivity test, P14 with Compound 5 exceeded 10¹⁰ Jones at both 1050 and 1350 nm, and P3 with Compound 6 exceeded 10⁸ Jones at both 1050 and 1350 nm. In this embodiment, not only the applications of the organic photoelectric component at a bias of −2 V were tested, but also the performance of the organic photoelectric component at a bias of −8 V was revealed. The dark current of P14 with Compound 5 was 8.2×10⁻⁵ A/cm² and the EQE was 5.6%, the responsivity was 0.047 A/W, and the detectivity was 9.3×10⁹ Jones at 1050 nm, and the EQE was 4.4%, the responsivity was 0.048 A/W, and the detectivity was 9.4×10⁹ Jones at 1350 nm. A breakthrough in the performance of materials with an absorbance onset value >1500 nm was achieved. The responsivity of Comparative Example 3 was <0.01 A/W, the EQE response of the material revealed in Comparative Example 1 ranged only from 300 to 850 nm, and this embodiment of the invention has expanded the EQE responsivity to above 1000 nm, indicating an improved performance of responsivity and expanded absorbance range. Moreover, when using the o-xylene as the solvent for the preparation of the organic photoelectric component, the excellent solubility would be favorable for the subsequent solution processing operation.

What is claimed is:

1. An organic semiconducting compound of the following formula: 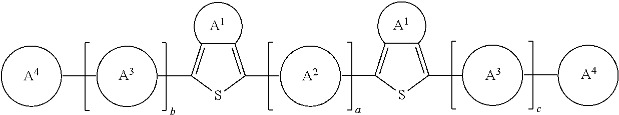
wherein,
A¹ is group selected from the following groups, 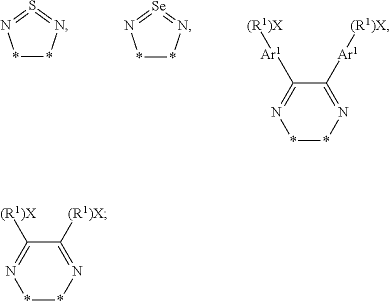
x is an integer between 0 and 5,
Ar¹ is aromatic ring or heteroaromatic ring group, which is monocyclic or polycyclic, and is unsubstituted or substituted with halogen atoms;
R¹ is selected from the group consisting of the following groups: hydrogen atom, halogen, cyano group, C1˜C30 linear alkyl, C3˜C30 branched alkyl, C1˜C30 silyl group, C2˜C30 ester group, C1˜C30 alkoxy, C1˜C30 thioalkyl, C1˜C30 haloalkyl, C2˜C30 alkene, C2˜C30 alkyne, C2˜C30 cyano-substituent alkyl, C1˜C30 nitro-substituent alkyl, C1˜C30 hydroxy-substituent alkyl, C3˜C30 keto-substituent alkyl;
A²-A⁴ are each aromatic ring or heteroaromatic ring group which is monocyclic or polycyclic; and
a, b, and c are an integer between 0 and 5.

2. The organic semiconducting compound according to claim 1, wherein A² is selected from the group composed of the following groups: 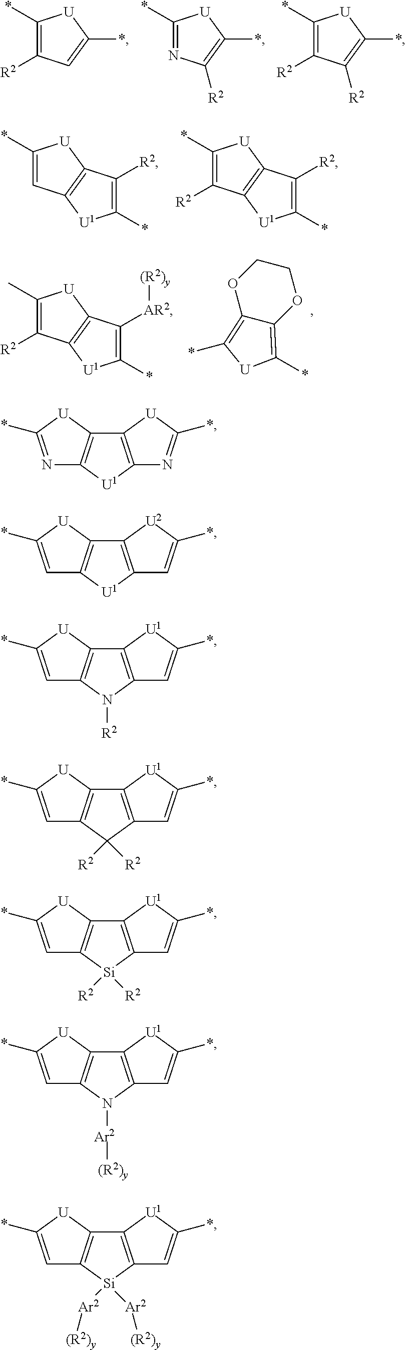 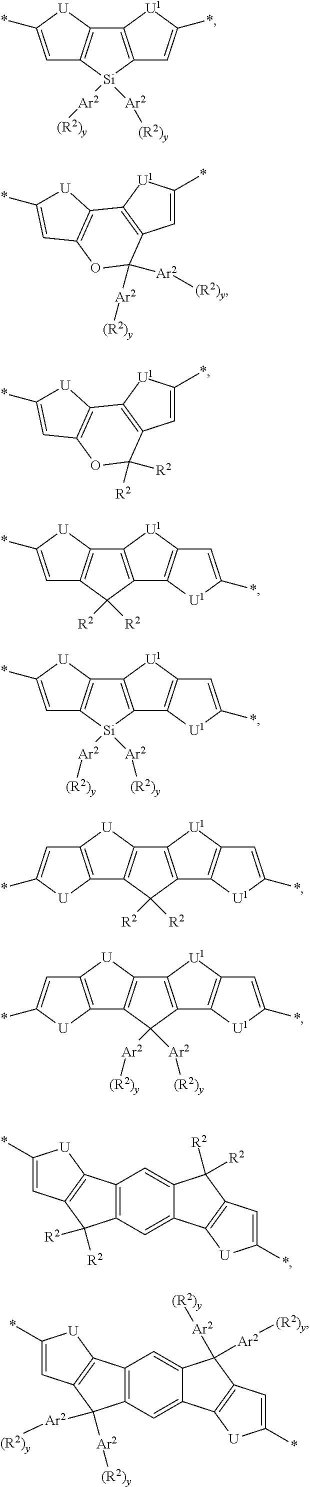 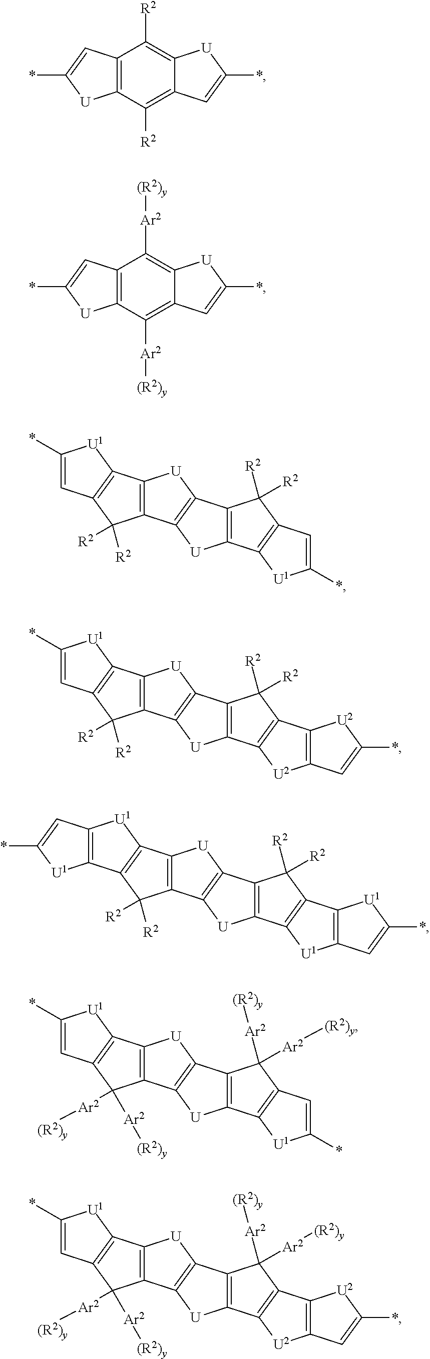 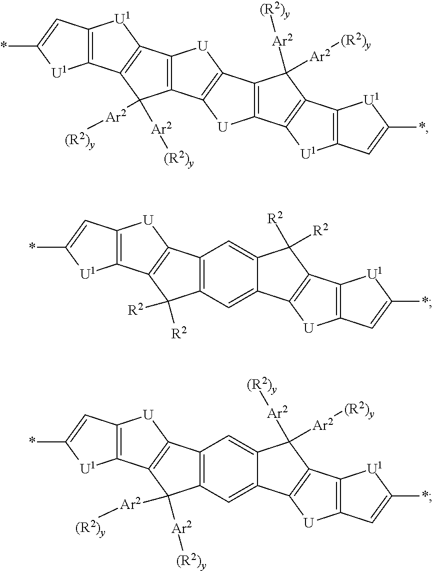
wherein
U, U¹ and U² are each O, S, or Sc;
y is an integer between 0 and 5;
Ar² is aromatic ring or heteroaromatic ring group, which is monocyclic or polycyclic, and is unsubstituted or substituted with halogen atoms; and
R² is selected from the group consisting of the following groups: hydrogen atom, halogen, cyano group, C1˜C30 linear alkyl, C3˜C30 branched alkyl, C1˜C30 silyl group, C2˜C30 ester group, C1˜C30 alkoxy, C1˜C30 thioalkyl, C1˜C30 haloalkyl, C2˜C30 alkene, C2˜C30 alkyne, C2˜C30 cyano-substituent alkyl, C1˜C30 nitro-substituent alkyl, C1˜C30 hydroxy-substituent alkyl, C3˜C30 keto-substituent alkyl.

3. The organic semiconducting compound according to claim 2, wherein A² is selected from the group composed of the following groups:

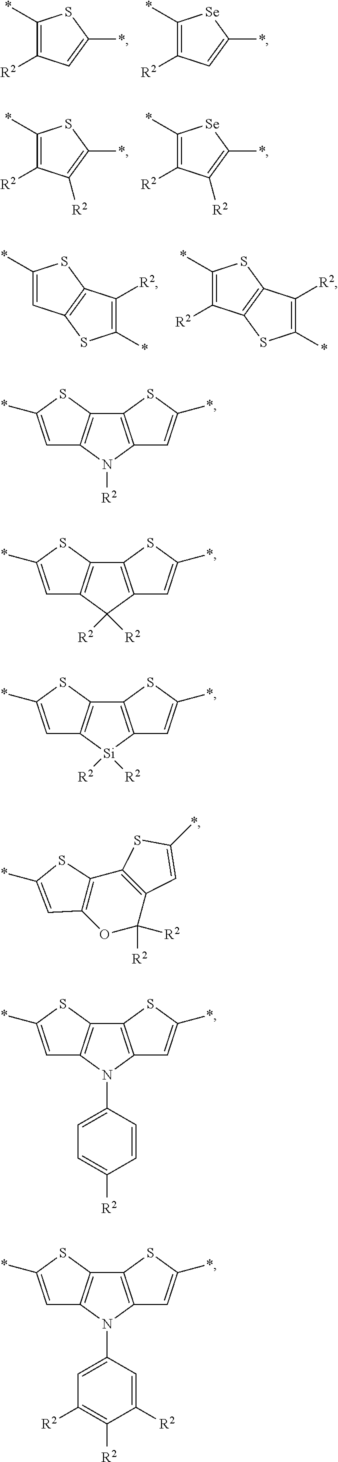

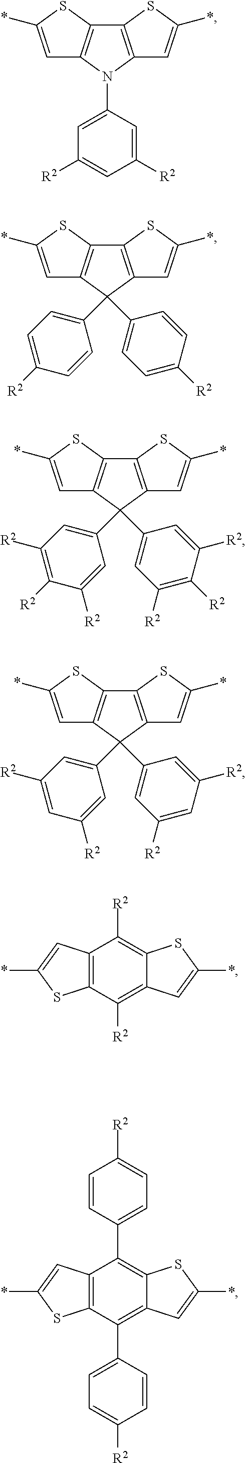

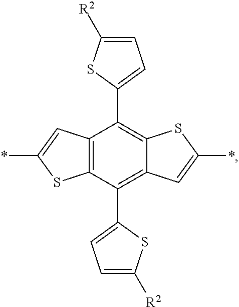

4. The organic semiconducting compound according to claim 1, wherein A³ is selected from the group consisting of the following groups: 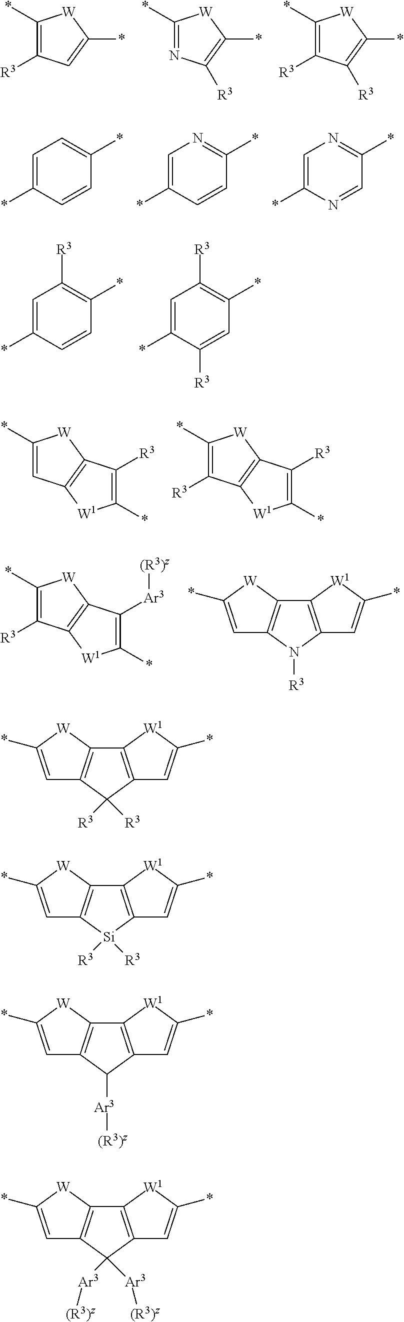 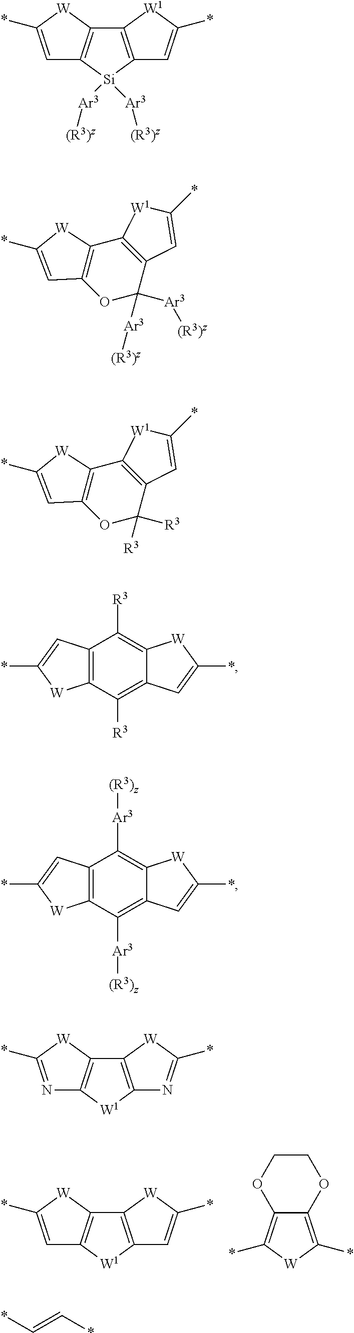
wherein,
W and W¹ are each O, S or Se;
z is integer between 0 and 5;
Ar³ is aromatic ring or heteroaromatic ring group, which is monocyclic or polycyclic, and is unsubstituted or substituted with halogen atoms; and
R³ is selected from the group consisting of the following groups: hydrogen atom, halogen, cyano group, C1˜C30 linear alkyl, C3˜C30 branched alkyl, C1˜C30 silyl group, C2˜C30 ester group, C1˜C30 alkoxy, C1˜C30 thioalkyl, C1˜C30 haloalkyl, C2˜C30 alkene, C2˜C30 alkyne, C2˜C30 cyano-substituent alkyl, C1˜C30 nitro-substituent alkyl, C1˜C30 hydroxy-substituent alkyl, C3˜C30 keto-substituent alkyl.

5. The organic semiconducting compound according to claim 4, wherein, A¹ is selected from the group consisting of the following groups:

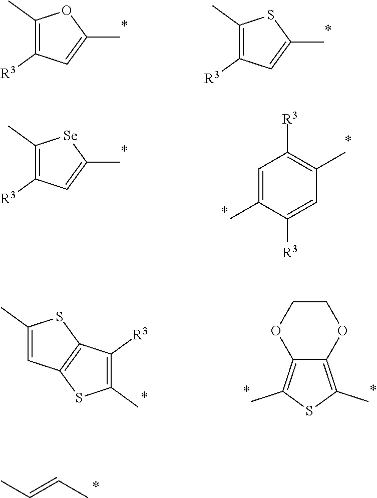

6. The organic semiconducting compound according to claim 1, wherein A⁴ is selected from the group consisting of the following groups: 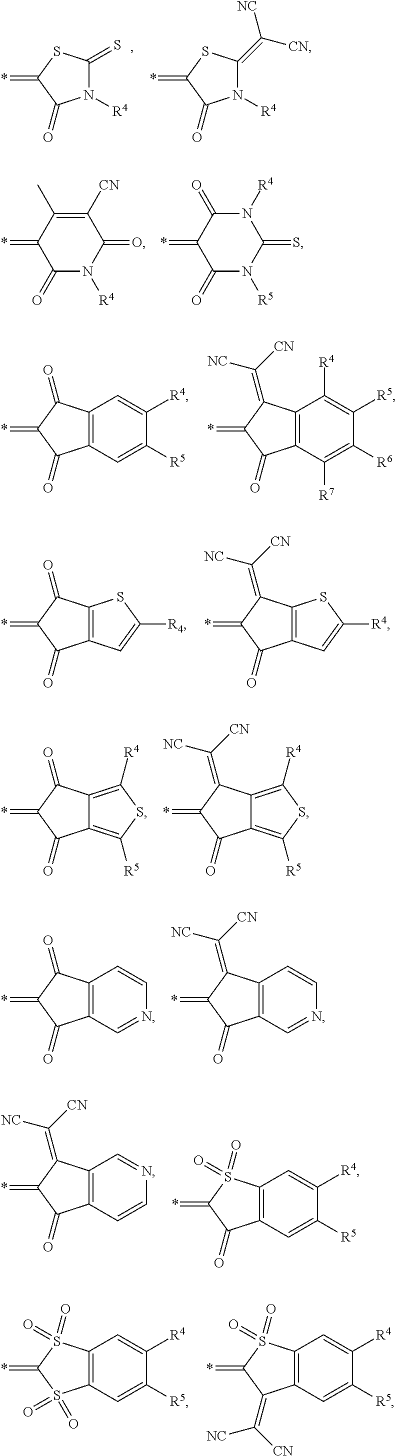 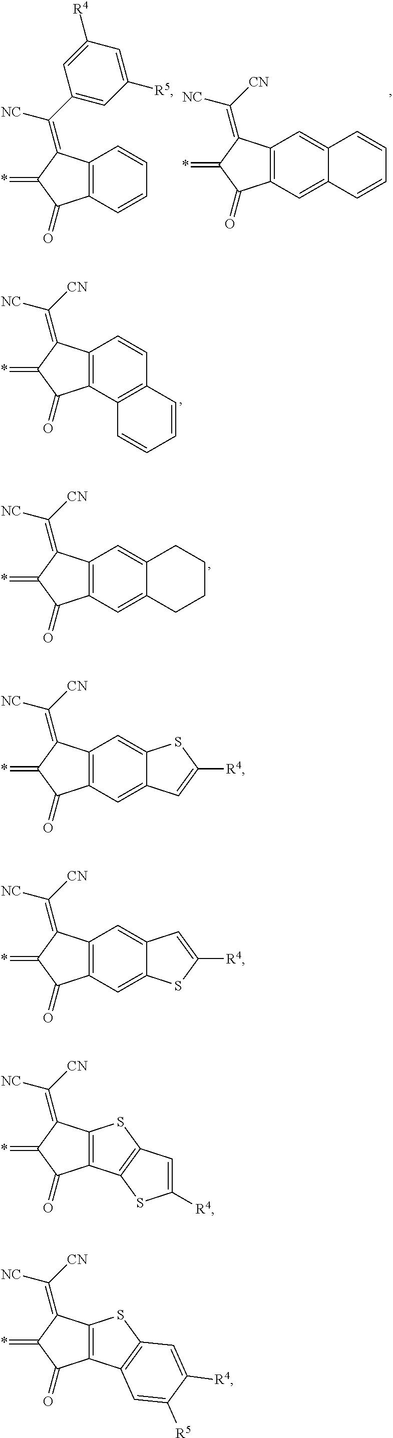 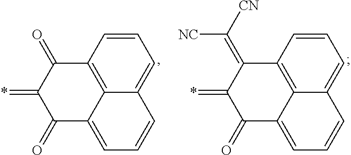 [SMILES: *=c1c(=O)c2cccc3cccc(c1=O)c32 ; *=C1C(=O)c2cccc3cccc(c23)C1=C(C#N)C#N]
and
R⁴˜R⁷ is selected from the group consisting of the following groups: hydrogen atom, halogen, cyano group, C1˜C30 linear alkyl, C3˜C30 branched alkyl, C1˜C30 silyl group, C2˜C30 ester group, C1˜C30 alkoxy, C1˜C30 thioalkyl, C1˜C30 haloalkyl, C2˜C30 alkene, C2˜C30 alkyne, C2˜C30 cyano-substituted alkyl, C1˜C30 nitro-substituted alkyl, C1˜C30 hydroxy-substituted alkyl, C3˜C30 keto-substituted alkyl.

7. An organic photoelectric component comprising:
a substrate;
an electrode module disposed on the substrate, which includes a first electrode and a second electrode; and
an active layer disposed between the first electrode and the second electrode, and the material of the active layer comprises at least one organic semiconducting compound according to claim 1;
wherein at least one of the first electrode and the second electrode is transparent or semi-transparent.

8. The organic photoelectronic component according to claim 7, wherein the first electrode, the active layer, and the second electrode are deposited on the substrate in the order from bottom to top.

9. The organic photoelectronic component according to claim 7, wherein the second electrode, the active layer, and the first electrode are deposited on the substrate in the order from bottom to top.

10. The organic photoelectronic component according to claim 7, wherein the active layer comprises at least one n-type organic semiconducting compound and at least one p-type organic semiconducting compound, and the n-type organic semiconducting compound is one of the organic semiconducting compounds according to claim 1.

11. The organic photoelectronic component according to claim 10, wherein the p-type organic semiconducting compound is selected from the group consisting of the following groups:

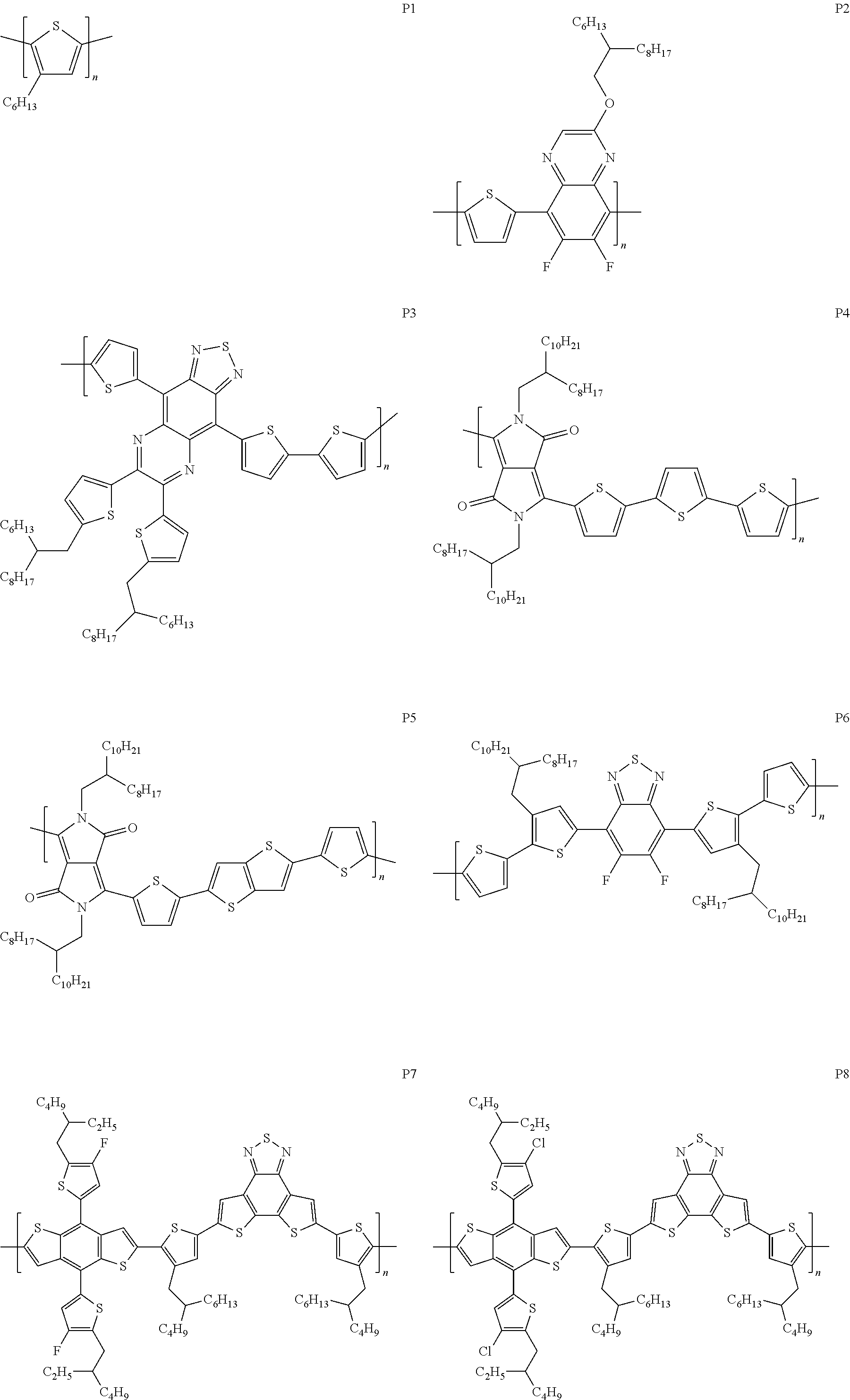

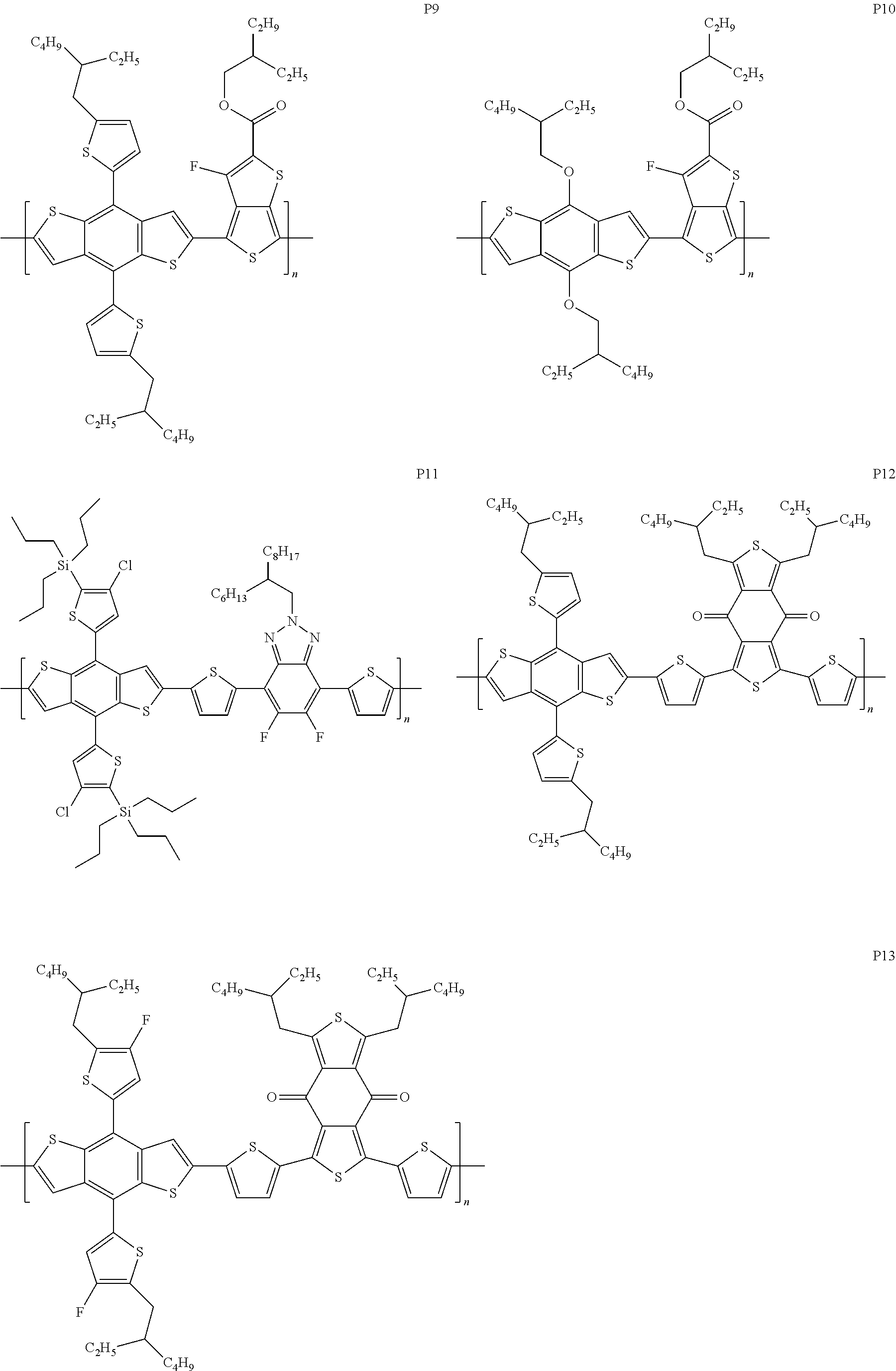

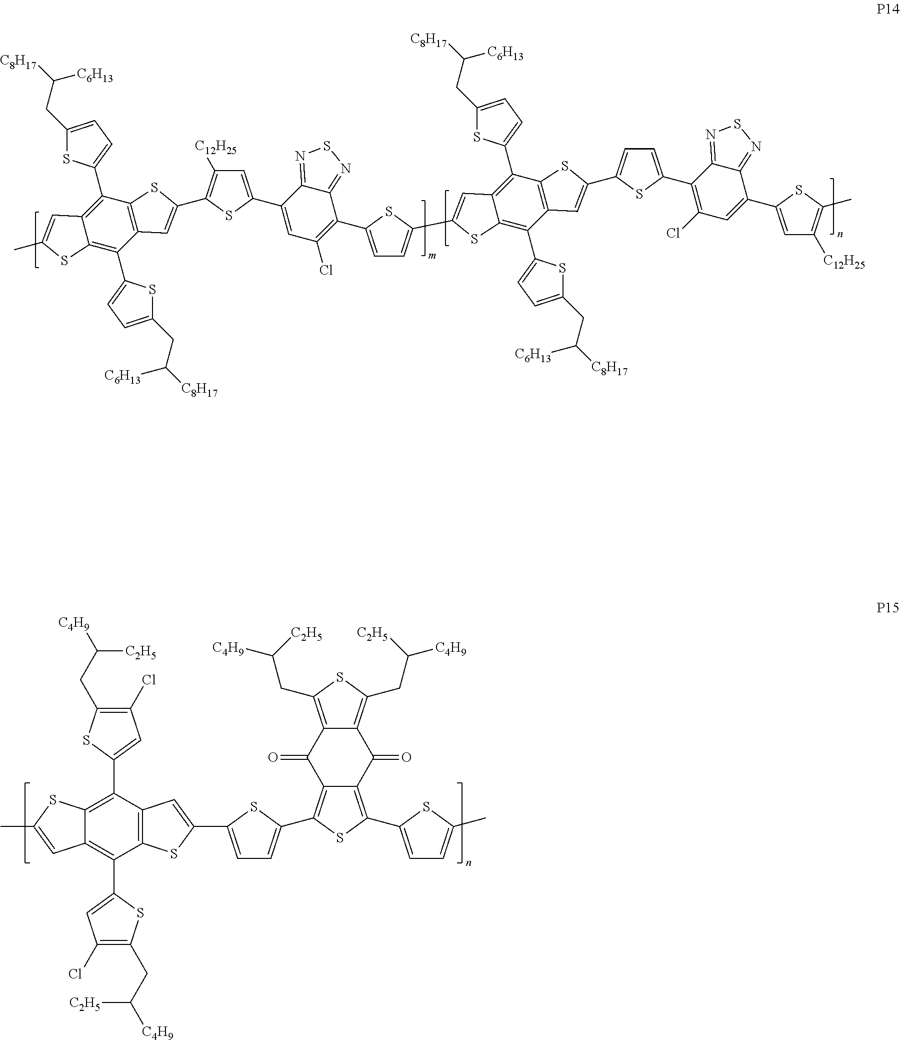

12. The organic photoelectronic component according to claim 7, which further comprises:
a first carrier transporting layer disposed between the first electrode and the active layer; and
a second carrier transporting layer disposed between the second electrode and the active layer.

13. The organic photoelectronic component according to claim 7, which further comprises:
a first carrier transport layer disposed between the second electrode and the active layer; and
a second carrier transfer layer disposed between the first electrode and the active layer.
